(19) United States
(12) Patent Application Publication
(10) Pub. No.: US 2016/0285006 A1
(43) Pub. Date: Sep. 29, 2016

(54) MATERIAL FOR AN ORGANIC ELECTROLUMINESCENT DEVICE AND ELECTROLUMINESCENT DEVICE USING THE SAME

(71) Applicant: SAMSUNG DISPLAY CO., LTD., Yongin-si (KR)

(72) Inventor: Hiroaki Itoi, Yokohama (JP)

(21) Appl. No.: 15/053,653

(22) Filed: Feb. 25, 2016

(30) Foreign Application Priority Data
Mar. 25, 2015 (JP) 2015-061923

(51) Int. Cl. H01L 51/00; C07D 495/16; C07D 491/16

(52) U.S. Cl. CPC H01L 51/0071; C07D 491/16; C07D 495/16; H01L 51/5056

(57) ABSTRACT

Embodiments of the present disclosure are directed toward a material for an organic electroluminescent device realizing a low driving voltage and high emission efficiency, and an organic electroluminescent device using the same. The material for an organic electroluminescent device is represented by Formula 1:

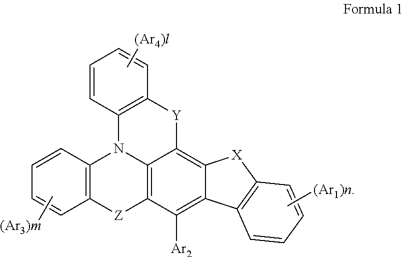

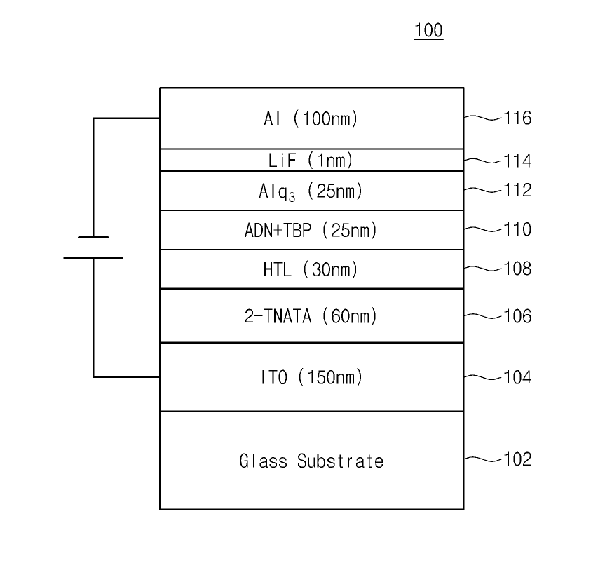

In Formula 1, X is selected from O, N or S, Y and Z are each independently selected from CAr₅Ar₆ or SiAr₇Ar₈, Ar₁ to Ar₈ are each independently selected from a substituted or unsubstituted alkyl group having 1 to 30 carbon atoms, a substituted or unsubstituted aryl group having 6 to 30 carbon atoms for forming a ring, a substituted or unsubstituted heteroaryl group having 5 to 30 carbon atoms for forming a ring, a silyl group, a halogen atom, deuterium, and hydrogen, and l to n are each independently an integer selected from 0 to 4.

### CROSS-REFERENCE TO RELATED APPLICATION

This application claims priority to and the benefit of Japanese Patent Application No. 2015-061923, filed on Mar. 25, 2015 in the Japan Patent Office, the entire content of which is incorporated herein by reference.

### BACKGROUND

1. Field

One or more aspects of embodiments of the present disclosure relate to a material for an organic electroluminescent device, and an organic electroluminescent device including the same.

2. Description of the Related Art

Organic electroluminescent (EL) displays are currently being actively developed. Unlike liquid crystal displays, etc. organic EL displays are so-called self-luminescent displays that function by recombining holes and electrons from an anode and a cathode in an emission layer to generate excitons. Light is emitted by a luminescent organic compound included in the emission layer.

An example organic EL device includes an anode, a hole transport layer on the anode, an emission layer on the hole transport layer, an electron transport layer on the emission layer and a cathode on the electron transport layer. Holes injected from the anode move via the hole transport layer to the emission layer. Electrons injected from the cathode move via the electron transport layer to the emission layer. When the holes and electrons injected into the emission layer are recombined, excitons may be generated in the emission layer. The organic EL device emits light using energy generated by the radiative decay of the excitons. Configurations of the organic EL device are not limited to the above example, and may be diversely modified.

When organic EL devices are used in display apparatuses, the organic EL devices should exhibit low driving voltages and high emission efficiencies. However, driving voltages are high and emission efficiencies are insufficient in many organic EL devices, for example, those in the blue and green emission regions. Methods of increasing the normalization and stabilization of the hole transport layer have been examined as strategies for reducing the driving voltage and increasing the efficiency of an organic EL device. Aromatic amine compounds have been used and are available as materials for use in an organic EL device. For example, an amine derivative substituted with a heteroaryl ring has been suggested as a useful material for an organic EL device. However, issues related to resolving the driving voltage and emission efficiency of the device remain.

Accordingly, an organic EL device having a lower driving voltage and higher emission efficiency is required at present. The improvement of emission efficiency in the blue and green emission regions is desirable. Developments on novel materials are required to realize organic EL devices with decreased driving voltages and increased emission efficiencies.

### SUMMARY

One or more aspects of embodiments of the present disclosure are directed toward a material for an organic EL device having a low driving voltage and high efficiency, and an organic EL device using the same.

Embodiments of the present disclosure relate to a material for an organic electroluminescent device and an organic electroluminescent device using the same, and in one embodiment, to a material for an organic electroluminescent device capable of being driven at a low voltage and having high emission efficiency in the blue emission region, as well as an organic electroluminescent device using the same.

An embodiment of the present disclosure provides a material for an organic EL device having a low driving voltage and high efficiency in the blue and/or green emission regions, and an organic EL device using the same in at least one stacking layer between an emission layer and an anode.

An embodiment of the present disclosure provides a material for an organic EL device, represented by the following Formula 1:

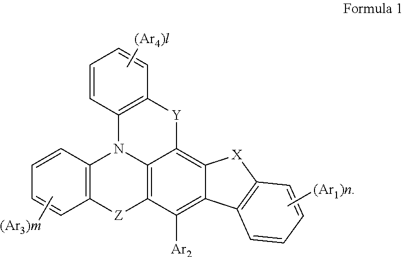

In Formula 1, X may be selected from O, N and S; Y and Z may each independently be selected from CAr₅Ar₆ and SiAr₇Ar₈; Ar₁ to Ar₈ may each independently be selected from a substituted or unsubstituted alkyl group having 1 to 30 carbon atoms, a substituted or unsubstituted aryl group having 6 to 30 carbon atoms for forming a ring, a substituted or unsubstituted heteroaryl group having 5 to 30 carbon atoms for forming a ring, a silyl group, a halogen atom, deuterium, and hydrogen, and l to n may each independently be an integer selected from 0 to 4.

The material for an organic EL device according to an embodiment of the present disclosure may be plane-immobilized (e.g., constrained to a planar structure) by making a crosslinking structure using quaternary carbon or silicon. The HOMO-LUMO gap may be increased and energy transfer may be restrained or reduced, thereby enabling high emission efficiency in the organic EL device. In some embodiments, remarkable effects may be attained in the blue and/or green emission regions.

In an embodiment of the present disclosure in Formula 1, X may be O; and Y and Z may each be CAr₅Ar₆.

In the material for an organic EL device according to an embodiment of the present disclosure, a benzofuroacridine-like moiety is plane-immobilized via a quaternary carbon, its HOMO-LUMO gap may be increased, and energy transfer may be restrained or reduced, thereby enabling high emission efficiency in the organic EL device. In some embodiments, remarkable effects may be attained in the blue and/or green emission regions.

In an embodiment of the present disclosure, Ar₁ to Ar₈ may each independently be selected from a phenyl group, a biphenyl group, a naphthyl group, a phenylnaphthyl group, a naphthylphenyl group and hydrogen.

In the material for an organic EL device according to an embodiment of the present disclosure, the molecular weight of the material for an organic EL device may be restrained or reduced when Ar₁ to Ar₈ are each independently selected from hydrogen, a phenyl group, a biphenyl group, a naphthyl group, a phenylnaphthyl group, and a naphthylphenyl group, and layer formation via evaporation may be conducted in the manufacturing of the organic EL device.

An embodiment of the present disclosure provides an organic EL device including the material for an organic EL device in at least one stacking layer between an emission layer and an anode.

The material for an organic EL device according to an embodiment of the present disclosure may be plane-immobilized by making a crosslinking structure using quaternary carbon or silicon. The HOMO-LUMO gap may be increased and energy transfer may be restrained or reduced, thereby enabling high emission efficiency in the organic EL device. In some embodiments, remarkable effects may be attained in the blue and/or green emission regions.

### BRIEF DESCRIPTION OF THE DRAWING

The accompanying drawing is included to enable further understanding of the present disclosure, and is incorporated in and constitutes a part of this specification. The drawing illustrates example embodiments of the present disclosure and, together with the description, serves to explain principles of the present disclosure.

The drawing is a schematic view showing an organic EL device 100 according to an embodiment of the present disclosure.

### DETAILED DESCRIPTION

Hereinafter, the material for an organic EL device and the organic EL device including the same according to an embodiment of the present disclosure will be described with reference to the accompanying drawing. The material for an organic EL device and the organic EL device including the same according to an embodiment may, however, be embodied in different forms and should not be construed as being limited to the embodiments set forth herein. In the drawings, like reference numerals refer to like elements or elements having like functions throughout, and repeated explanation thereof will not be provided.

The thickness of layers, films, panels, regions, etc., may be exaggerated in the drawings for clarity. It will be understood that when an element such as a layer, film, region, or substrate is referred to as being “on” another element, it can be directly on the other element or intervening elements may also be present. In contrast, when an element is referred to as being “directly on” another element, no intervening elements are present.

The material for an organic EL device according to an embodiment of the present disclosure may include derivatives represented by the following Formula 1:

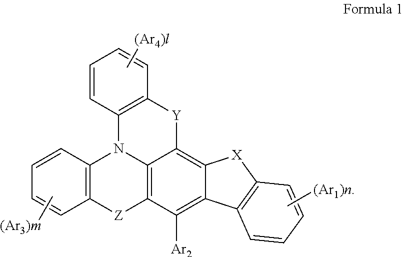

In Formula 1, X may be selected from O, N and S; and Y and Z may each independently be selected from CAr₅Ar₆ and SiAr₇Ar₈. Ar₁ to Ar₈ may each independently be selected from a substituted or unsubstituted alkyl group having 1 to 30 carbon atoms, a substituted or unsubstituted aryl group having 6 to 30 carbon atoms for forming a ring, a substituted or unsubstituted heteroaryl group having 5 to 30 carbon atoms for forming a ring, a silyl group, a halogen atom, deuterium, and hydrogen. l to n may each independently be an integer selected from 0 to 4. As used herein, “atoms for forming a ring” may refer to “ring-forming atoms”.

Non-limiting examples of the alkyl group having 1 to 30 carbon atoms used as Ar₁ to Ar₈ may include a methyl group, an ethyl group, an n-propyl group, an isopropyl group, an n-butyl group, an s-butyl group, a t-butyl group, an i-butyl group, a 2-ethylbutyl group, a 3,3-dimethylbutyl group, an n-pentyl group, an i-pentyl group, a neopentyl group, a t-pentyl group, a cyclopentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, a cyclohexyl group, a 4-methylcyclohexyl group, a 4-t-butylcyclohexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, a cyclooctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldodecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-icosyl group, a 2-ethylicosyl group, a 2-butylicosyl group, a 2-hexylicosyl group, a 2-octylicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, etc.

Non-limiting examples of the substituted or unsubstituted aryl group having 6 to 30 carbon atoms for forming a ring used as Ar₁ to Ar₈ may include a phenyl group, a naphthyl group, an anthracenyl group, a phenanthryl group, a biphenyl group, a terphenyl group, a quaterphenyl group, a quinquephenyl group, a sexiphenyl group, a fluorenyl group, a triphenylene group, a biphenylene group, a pyrenyl group, a benzofluoranthenyl group, a chrysenyl group, etc.

Non-limiting examples of the substituted or unsubstituted heteroaryl group having 5 to 30 carbon atoms for forming a ring used as Ar₁ to Ar₈ may include a benzothiazolyl group, a thiophenyl group, a thienothiophenyl group, a thienothienothiophenyl group, a benzothiophenyl group, a benzofuryl group, a dibenzothiophenyl group, a dibenzofuryl group, an N-arylcarbazolyl group, an N-heteroarylcarbazolyl group, an N-alkylcarbazolyl group, a phenoxazyl group, a phenothiazyl group, a pyridyl group, a pyrimidyl group, a triazinyl group, a quinolinyl group, a quinoxalyl group, etc.

In an embodiment of the present disclosure, Ar₁ to Ar₈ may each independently be selected from a phenyl group, a biphenyl group, a naphthyl group, a phenylnaphthyl group, a naphthylphenyl group, and hydrogen. When Ar₁ to Ar₈ are selected from the above substituents, the molecular weight of the material for an organic EL device may be restrained or reduced, and a layer may be formed by evaporation in the manufacturing of an organic EL device.

The material for an organic EL device according to an embodiment of the present disclosure may be plane-immobilized (e.g., constrained to a planar structure) by making a crosslinking structure using quaternary carbon or silicon. The HOMO-LUMO gap may be increased and energy transfer may be restrained or reduced, thereby enabling high emission efficiency in the organic EL device. In some embodiments, remarkable effects may be attained in the blue and/or green emission regions.

The material for an organic EL device according to the present disclosure may be represented by one selected from the following Formulae 2 to 5:

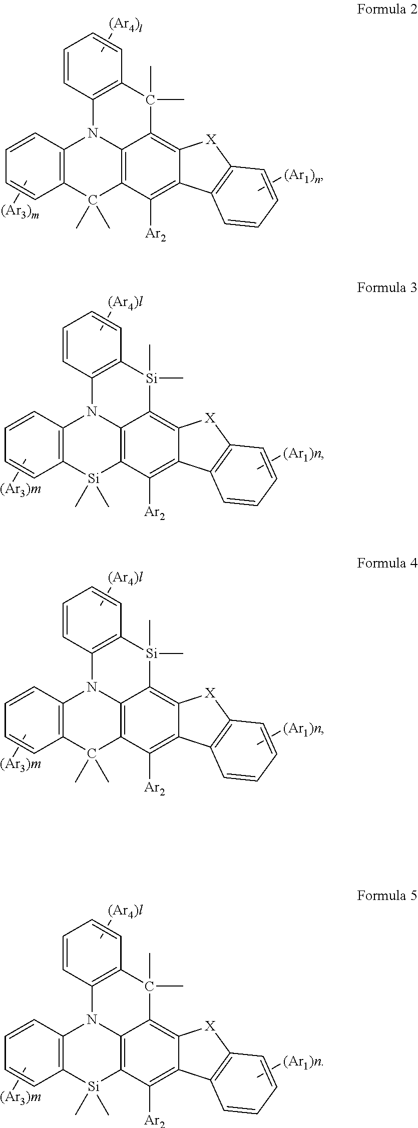

In Formulae 2 to 5, X may be selected from O, N and S. Ar₁ to Ar₄ may each independently be selected from a substituted or unsubstituted alkyl group having 1 to 30 carbon atoms, a substituted or unsubstituted aryl group having 6 to 30 carbon atoms for forming a ring, a substituted or unsubstituted heteroaryl group having 5 to 30 carbon atoms for forming a ring, a silyl group, a halogen atom, deuterium, and hydrogen. l to n may each independently be an integer selected from 0 to 4.

Ar₁ to Ar₄ of Formulae 2 to 5 may each be independently selected from the same groups as Ar₁ to Ar₈ of Formula 1, and detailed descriptions thereof will not be provided again.

In the material for an organic EL device of Formulae 1 to 5, a plurality of Ar₁ to Ar₈ may combine to (e.g., couple with) each other to form a saturated or unsaturated ring.

In an embodiment of the present disclosure, a compound in which a benzofuroacridine-like moiety is crosslinked via quaternary carbon may be more easily synthesized and have a lower driving voltage and higher emission efficiency when compared to a compound in which X is selected from N and S and/or a compound in which Y and Z are SiAr₇Ar₈. As used herein, the term “benzofuroacridine-like moiety” refers to a moiety with a structure similar to that of benzofuroacridine, as illustrated by the lower five fused rings in, for example, Formula 2.

In an embodiment of the present disclosure, the material for an organic EL device represented by Formula 1 may be selected from the following Compounds 1 to 7:

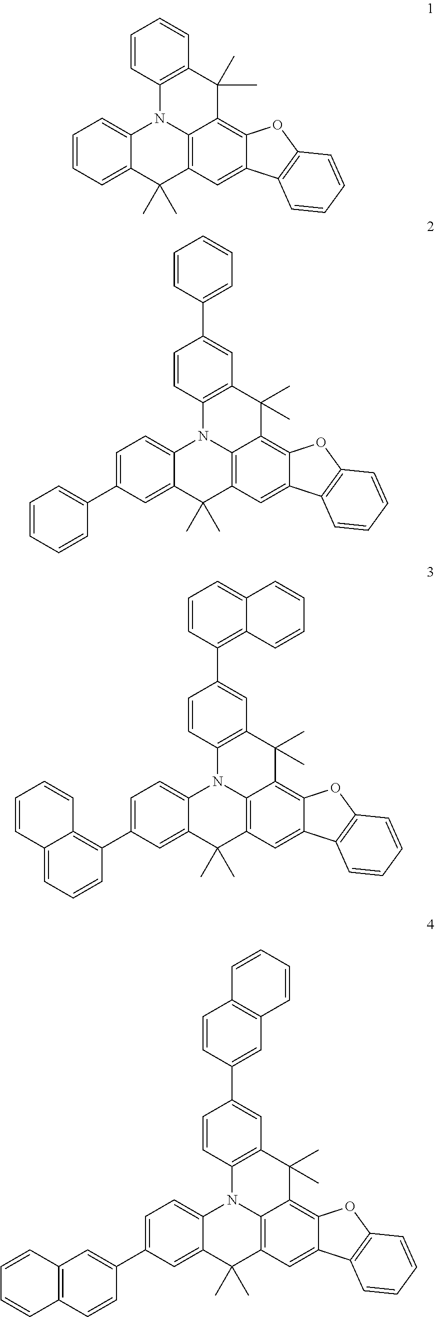

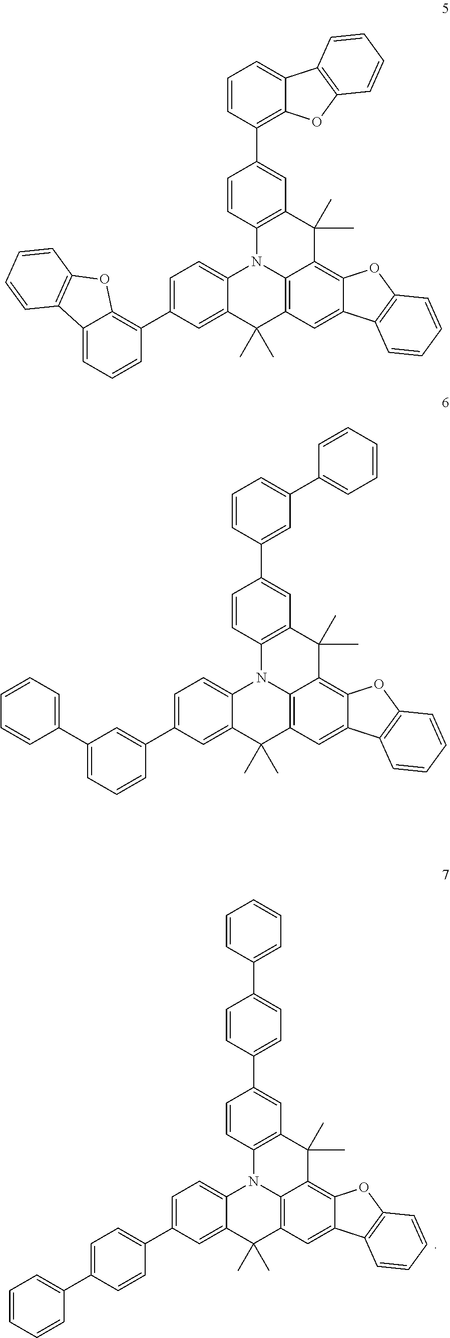 [SMILES: CC1(C)c2cc(-c3cccc4c3oc3ccccc34)ccc2N2c3ccc(-c4cccc5c4oc4ccccc45)cc3C(C)(C)c3c2c1cc1c3oc2ccccc21 ; CC1(C)c2cc(-c3cccc(-c4ccccc4)c3)ccc2N2c3ccc(-c4cccc(-c5ccccc5)c4)cc3C(C)(C)c3c2c1cc1c3oc2ccccc21 ; CC1(C)c2cc(-c3ccc(-c4ccccc4)cc3)ccc2N2c3ccc(-c4ccc(-c5ccccc5)cc4)cc3C(C)(C)c3c2c1cc1c3oc2ccccc21]

In an embodiment of the present disclosure, the material for an organic EL device represented by Formula 1 may be selected from the following Compounds 8 to 14:

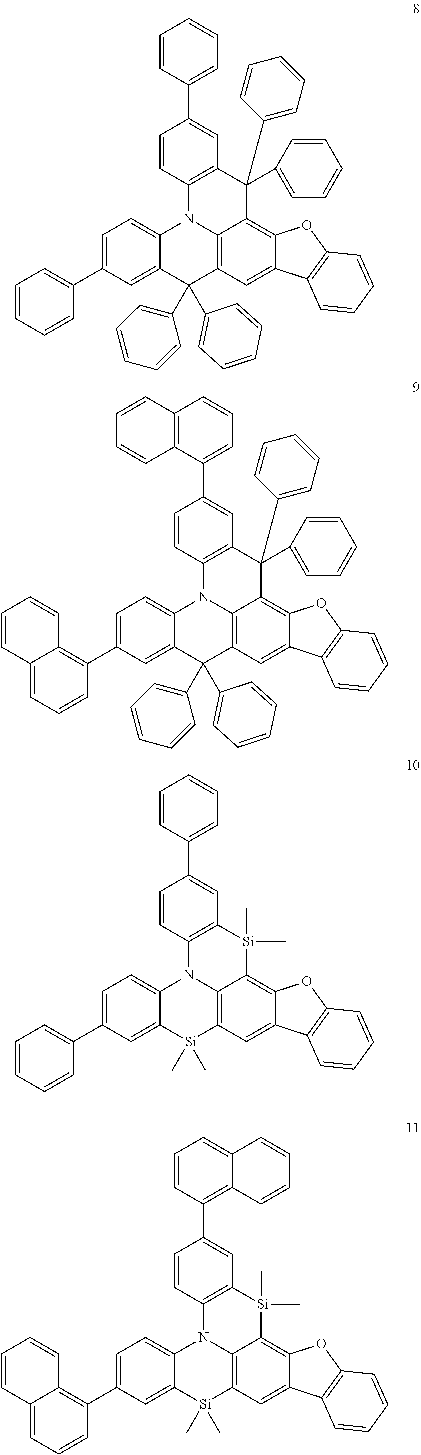

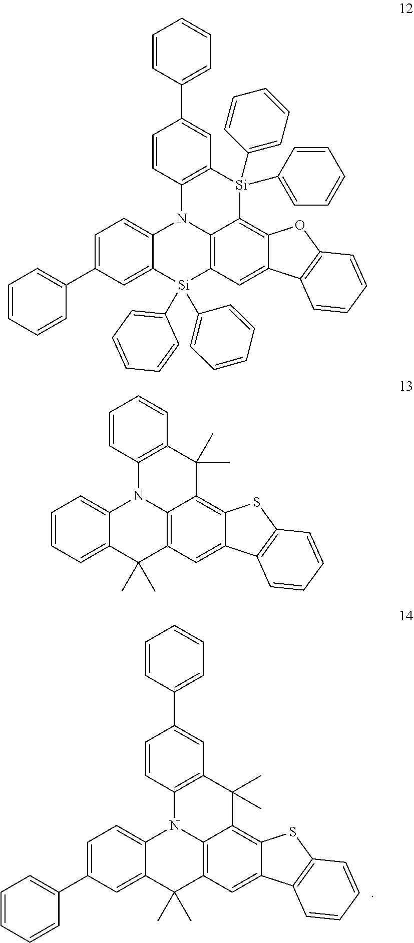

In an embodiment of the present disclosure, the material for an organic EL device represented by Formula 1 may be selected from the following Compounds 15 to 20:

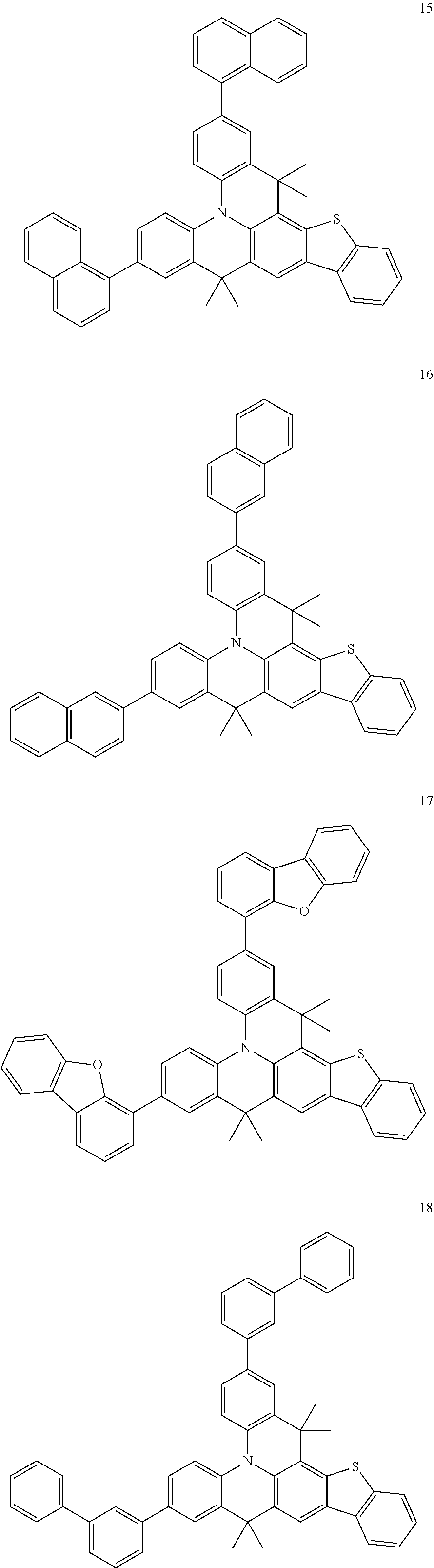 [SMILES: CC1(C)c2cc(-c3cccc4ccccc34)ccc2N2c3ccc(-c4cccc5ccccc45)cc3C(C)(C)c3c2c1cc1c3sc2ccccc21 ; CC1(C)c2cc(-c3ccc4ccccc4c3)ccc2N2c3ccc(-c4ccc5ccccc5c4)cc3C(C)(C)c3c2c1cc1c3sc2ccccc21 ; CC1(C)c2cc(-c3cccc4c3oc3ccccc34)ccc2N2c3ccc(-c4cccc5c4oc4ccccc45)cc3C(C)(C)c3c2c1cc1c3sc2ccccc21 ; CC1(C)c2cc(-c3cccc(-c4ccccc4)c3)ccc2N2c3ccc(-c4cccc(-c5ccccc5)c4)cc3C(C)(C)c3c2c1cc1c3sc2ccccc21]

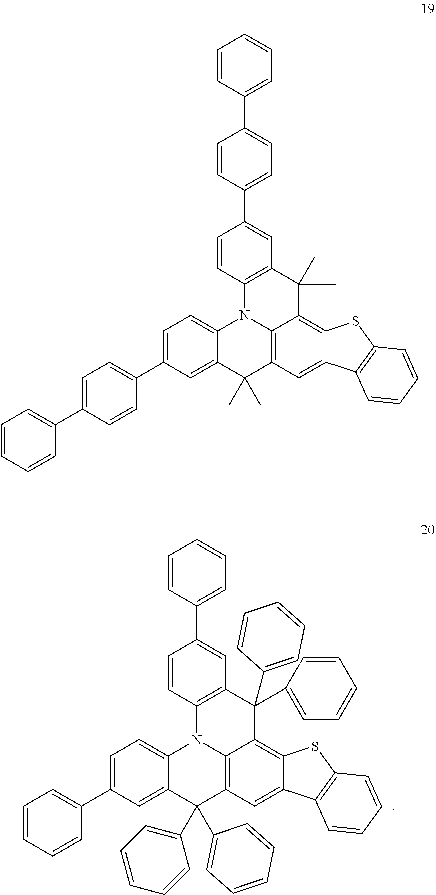 [SMILES: CC1(C)c2cc(-c3ccc(-c4ccccc4)cc3)ccc2N2c3ccc(-c4ccc(-c5ccccc5)cc4)cc3C(C)(C)c3c2c1cc1c3sc2ccccc21 ; c1ccc(-c2ccc3c(c2)C(c2ccccc2)(c2ccccc2)c2cc4c(sc5ccccc54)c4c2N3c2ccc(-c3ccccc3)cc2C4(c2ccccc2)c2ccccc2)cc1]

In an embodiment of the present disclosure, the material for an organic EL device represented by Formula 1 may be selected from the following Compounds 21 to 27:

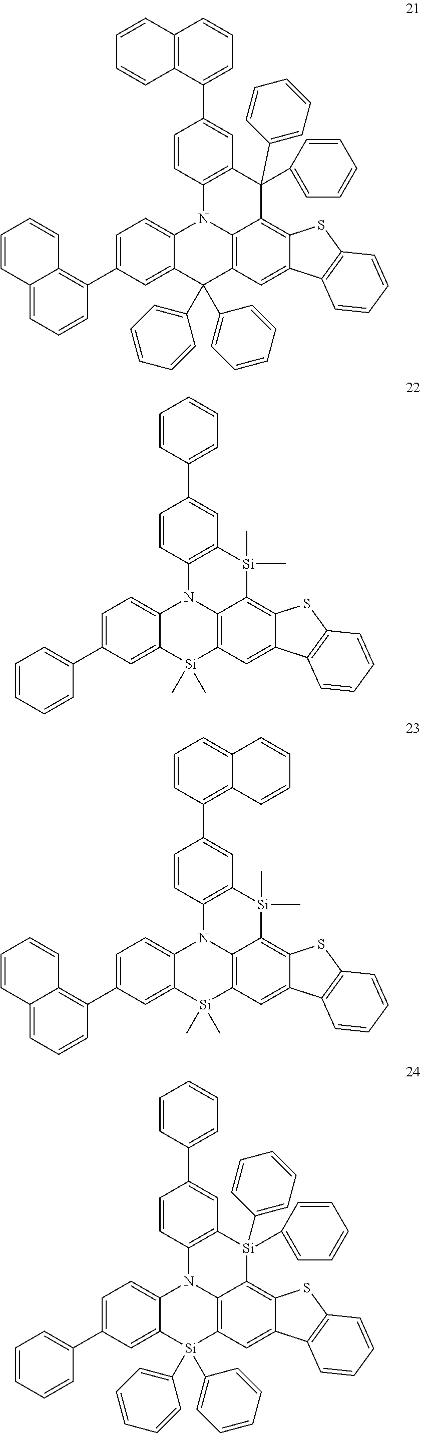

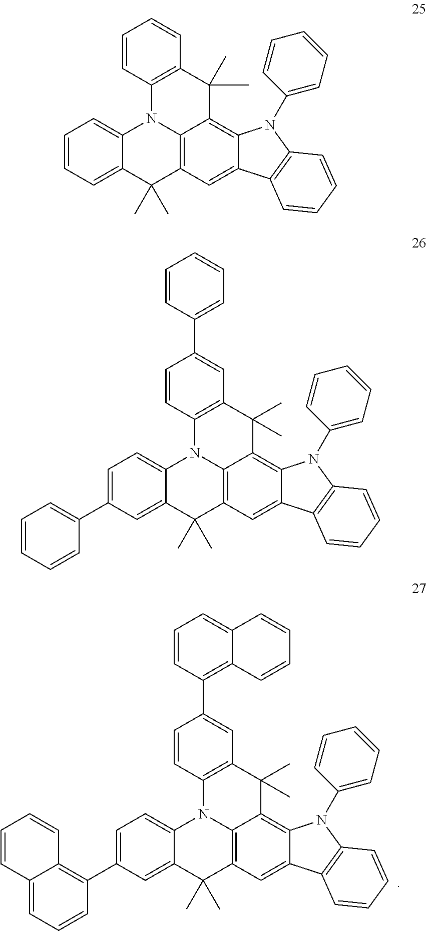 [SMILES: CC1(C)c2ccccc2N2c3ccccc3C(C)(C)c3c2c1cc1c2ccccc2n(-c2ccccc2)c31 ; CC1(C)c2cc(-c3ccccc3)ccc2N2c3ccc(-c4ccccc4)cc3C(C)(C)c3c2c1cc1c2ccccc2n(-c2ccccc2)c31 ; CC1(C)c2cc(-c3cccc4ccccc34)ccc2N2c3ccc(-c4cccc5ccccc45)cc3C(C)(C)c3c2c1cc1c2ccccc2n(-c2ccccc2)c31]

In an embodiment of the present disclosure, the material for an organic EL device represented by Formula 1 may be selected from the following Compounds 28 to 33:

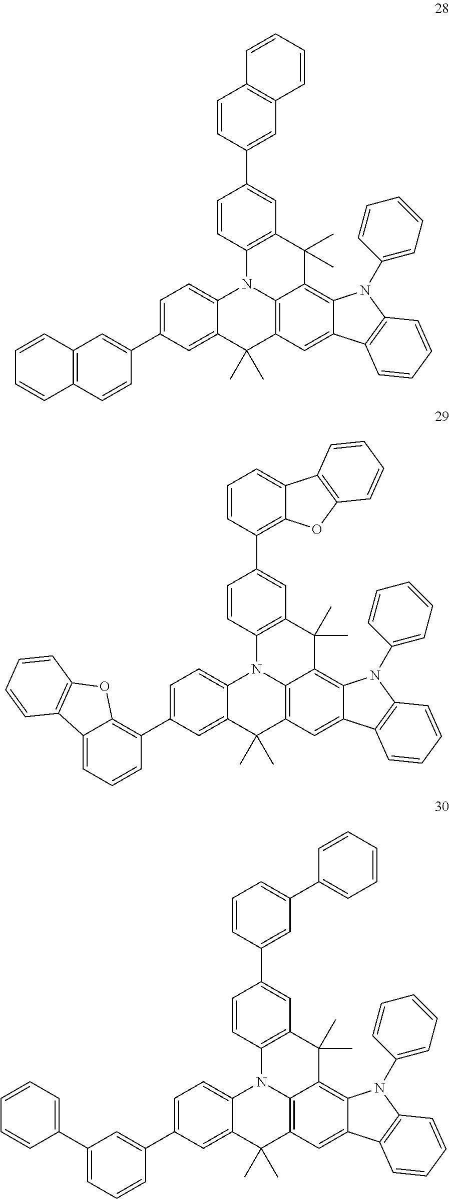 [SMILES: CC1(C)c2cc(-c3ccc4ccccc4c3)ccc2N2c3ccc(-c4ccc5ccccc5c4)cc3C(C)(C)c3c2c1cc1c2ccccc2n(-c2ccccc2)c31 ; CC1(C)c2cc(-c3cccc4c3oc3ccccc34)ccc2N2c3ccc(-c4cccc5c4oc4ccccc45)cc3C(C)(C)c3c2c1cc1c2ccccc2n(-c2ccccc2)c31 ; CC1(C)c2cc(-c3cccc(-c4ccccc4)c3)ccc2N2c3ccc(-c4cccc(-c5ccccc5)c4)cc3C(C)(C)c3c2c1cc1c2ccccc2n(-c2ccccc2)c31]

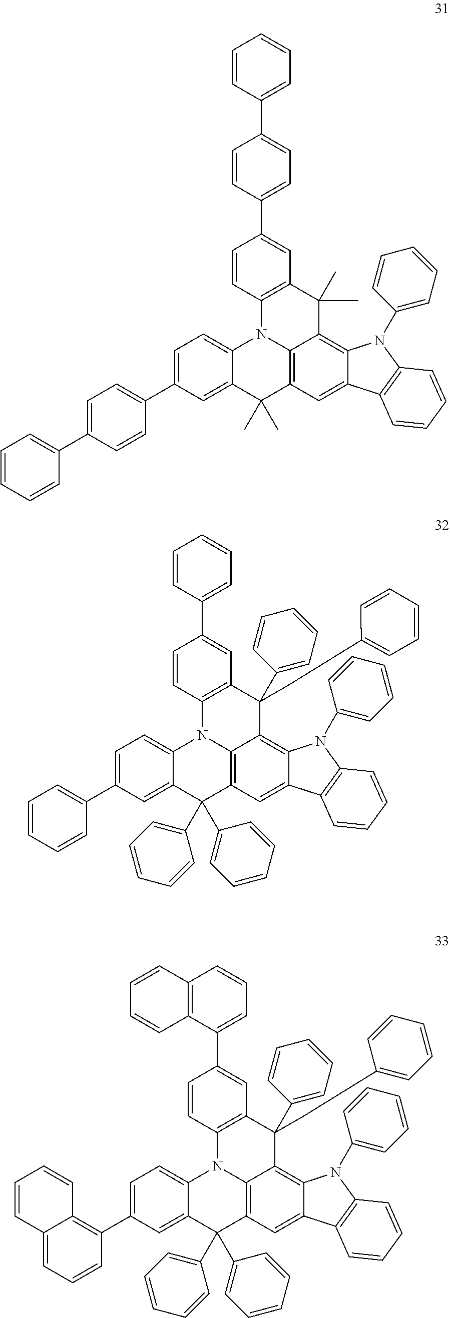 [SMILES: CC1(C)c2cc(-c3ccc(-c4ccccc4)cc3)ccc2N2c3ccc(-c4ccc(-c5ccccc5)cc4)cc3C(C)(C)c3c2c1cc1c2ccccc2n(-c2ccccc2)c31 ; c1ccc(CC2(c3ccccc3)c3cc(-c4ccccc4)ccc3N3c4ccc(-c5ccccc5)cc4C(c4ccccc4)(c4ccccc4)c4cc5c6ccccc6n(-c6ccccc6)c5c2c43)cc1 ; c1ccc(CC2(c3ccccc3)c3cc(-c4cccc5ccccc45)ccc3N3c4ccc(-c5cccc6ccccc56)cc4C(c4ccccc4)(c4ccccc4)c4cc5c6ccccc6n(-c6ccccc6)c5c2c43)cc1]

In an embodiment of the present disclosure, the material for an organic EL device represented by Formula 1 may be selected from the following Compounds 34 to 39:

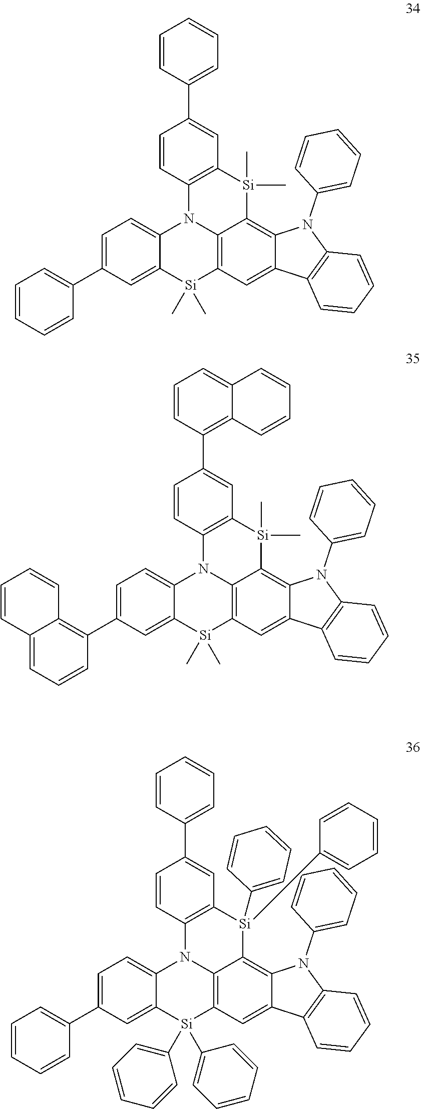 [SMILES: C[Si]1(C)c2cc(-c3ccccc3)ccc2N2c3ccc(-c4ccccc4)cc3[Si](C)(C)c3c2c1cc1c2ccccc2n(-c2ccccc2)c31 ; C[Si]1(C)c2cc(-c3cccc4ccccc34)ccc2N2c3ccc(-c4cccc5ccccc45)cc3[Si](C)(C)c3c2c1cc1c2ccccc2n(-c2ccccc2)c31 ; c1ccc(CC[Si]2(c3ccccc3)c3cc(-c4ccccc4)ccc3N3c4ccc(-c5ccccc5)cc4[Si](c4ccccc4)(c4ccccc4)c4cc5c6ccccc6n(-c6ccccc6)c5c2c43)cc1]

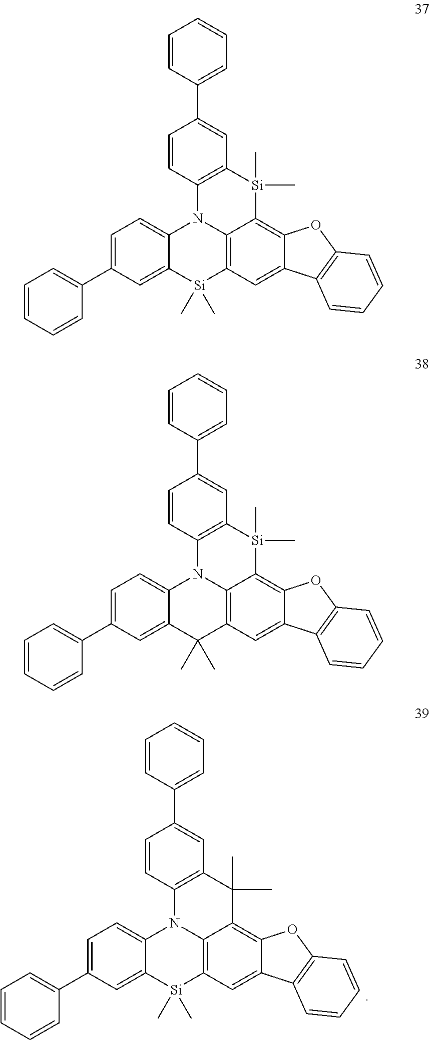 [SMILES: C[Si]1(C)c2cc(-c3ccccc3)ccc2N2c3ccc(-c4ccccc4)cc3[Si](C)(C)c3c2c1cc1c3oc2ccccc21 ; CC1(C)c2cc(-c3ccccc3)ccc2N2c3ccc(-c4ccccc4)cc3[Si](C)(C)c3c2c1cc1c3oc2ccccc21 ; CC1(C)c2cc(-c3ccccc3)ccc2N2c3ccc(-c4ccccc4)cc3[Si](C)(C)c3cc4c(oc5ccccc54)c1c32]

The material for an organic EL device may be used in at least one stacking layer between an emission layer and an anode. The material for an organic EL device may be plane-immobilized by making a cross-linked structure via quaternary carbon or silicon, the HOMO-LUMO gap may be increased, and energy transfer may be restrained or reduced, thereby enabling high emission efficiency in the organic EL device.

### Organic EL Device

An organic EL device using the material for an organic EL device according to the present disclosure will be described in more detail. The drawing is a schematic diagram illustrating an organic EL device 100 according to an embodiment of the present disclosure. The organic EL device 100 may include, for example, a substrate 102, an anode 104, a hole injection layer 106, a hole transport layer 108, an emission layer 110, an electron transport layer 112, an electron injection layer 114 and a cathode 116. In one embodiment, the material for an organic EL device according to an embodiment of the present disclosure may be used in at least one stacking layer between the emission layer and the anode.

An embodiment in which the material for an organic EL device according to the present disclosure is used in the hole transport layer 108 will be described in more detail. The substrate 102 may be a transparent glass substrate, a semiconductor substrate formed using silicon, a flexible substrate of a resin, etc. The anode 104 may be on the substrate 102 and may be formed using indium tin oxide (ITO), indium zinc oxide (IZO), etc. The hole injection layer 106 may be on the anode 104 and may be formed using 4,4′,4″-tris[2-naphthyl(phenyl)amino]triphenylamine (2-TNATA), N,N,N′,N′-tetrakis(3-methylphenyl)-3,3′-dimethylbenzidine (HMTPD), etc. The hole transport layer 108 may be formed on the hole injection layer 106 using the material for an organic EL device according to the present disclosure. In one embodiment, the thickness of the hole transport layer 108 may be in a range of about 3 nm to about 100 nm.

The emission layer 110 may be formed on the hole transport layer 108, may include condensed polycyclic aromatic derivatives, and may be selected from an anthracene derivative, a pyrene derivative, a fluoranthene derivative, a chrysene derivative, a benzoanthracene derivative and a triphenylene derivative. The emission layer 110 may include an anthracene derivative and/or a pyrene derivative. As the anthracene derivative used in the emission layer 110, a compound represented by the following Formula 6 may be used:

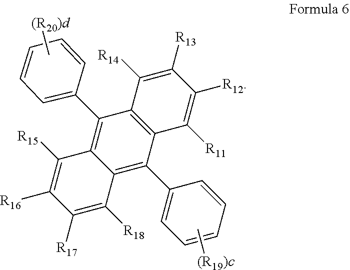

In Formula 6, R₁₁ to R₂₀ may each independently be selected from a substituted or unsubstituted aryl group having 6 to 30 carbon atoms for forming a ring, a substituted or unsubstituted heteroaryl group having 1 to 30 carbon atoms for forming a ring, an alkyl group having 1 to 15 carbon atoms, a silyl group, a halogen atom, hydrogen, and deuterium. c and d may each independently be an integer selected from 0 to 5. A plurality of adjacent R₁₁ to R₂₀ may make a bond to form a saturated or unsaturated ring.

Non-limiting examples of the substituted or unsubstituted aryl group having 6 to 30 carbon atoms for forming a ring used as R₁₁ to R₂₀ may include a phenyl group, a naphthyl group, an anthracenyl group, a phenanthryl group, a biphenyl group, a terphenyl group, a quaterphenyl group, a quinquephenyl group, a sexiphenyl group, a fluorenyl group, a triphenylene group, a biphenylene group, a pyrenyl group, a benzofluoranthenyl group, a chrysenyl group, etc.

Non-limiting examples of the substituted or unsubstituted heteroaryl group having 1 to 30 carbon atoms for forming a ring used as R₁₁ to R₂₀ may include a benzothiazolyl group, a thiophenyl group, a thienothiophenyl group, a thienothienothiophenyl group, a benzothiophenyl group, a benzofuryl group, a dibenzothiophenyl group, a dibenzofuryl group, an N-aryl carbazolyl group, an N-heteroarylcarbazolyl group, an N-alkylcarbazolyl group, a phenoxazyl group, a phenothiazyl group, a pyridyl group, a pyrimidyl group, a triazinyl group, a quinolinyl group, a quinoxalyl group, etc.

Non-limiting examples of the alkyl group having 1 to 15 carbon atoms used as R₁₁ to R₂₀ may include a methyl group, an ethyl group, a propyl group, an isopropyl group, an n-butyl group, an s-butyl group, an isobutyl group, a t-butyl group, an n-pentyl group, an n-hexyl group, an n-heptyl group, an n-octyl group, a hydroxymethyl group, a 1-hydroxyethyl group, a 2-hydroxyethyl group, a 2-hydroxyisobutyl group, a 1,2-dihydroxyethyl group, a 1,3-dihydroxyisopropyl group, a 2,3-dihydroxy-t-butyl group, a 1,2,3-trihydroxypropyl group, a chloromethyl group, a 1-chloroethyl group, a 2-chloroethyl group, a 2-chloroisobutyl group, a 1,2-dichloroethyl group, a 1,3-dichloroisopropyl group, a 2,3-dichloro-t-butyl group, a 1,2,3-trichloropropyl group, a bromomethyl group, a 1-bromoethyl group, a 2-bromoethyl group, a 2-bromoisobutyl group, a 1,2-dibromoethyl group, a 1,3-dibromoisopropyl group, a 2,3-dibromo-t-butyl group, a 1,2,3-tribromopropyl group, an iodomethyl group, a 1-iodoethyl group, a 2-iodoethyl group, a 2-iodoisobutyl group, a 1,2-diiodoethyl group, a 1,3-diiodoisopropyl group, a 2,3-diiodo-t-butyl group, a 1,2,3-triiodopropyl group, an aminomethyl group, a 1-aminoethyl group, a 2-aminoethyl group, a 2-aminoisobutyl group, a 1,2-diaminoethyl group, a 1,3-diaminoisopropyl group, a 2,3-diamino-t-butyl group, a 1,2,3-triaminopropyl group, a cyanomethyl group, a 1-cyanoethyl group, a 2-cyanoethyl group, a 2-cyanoisobutyl group, a 1,2-dicyanoethyl group, a 1,3-dicyanoisopropyl group, a 2,3-dicyano-t-butyl group, a 1,2,3-tricyanopropyl group, a nitromethyl group, a 1-nitroethyl group, a 2-nitroethyl group, a 2-nitroisobutyl group, a 1,2-dinitroethyl group, a 1,3-dinitroisopropyl group, a 2,3-dinitro-t-butyl group, a 1,2,3-trinitropropyl group, a cyclopropyl group, a cyclobutyl group, a cyclopentyl group, a cyclohexyl group, a 4-methylcyclohexyl group, a 1-adamantyl group, a 2-adamantyl group, a 1-norbornyl group, a 2-norbornyl group, etc.

Non-limiting examples of the anthracene derivative used in the emission layer 110 of the organic EL device according to an embodiment of the present disclosure may include compounds represented by the following Formulae a-1 to a-6:

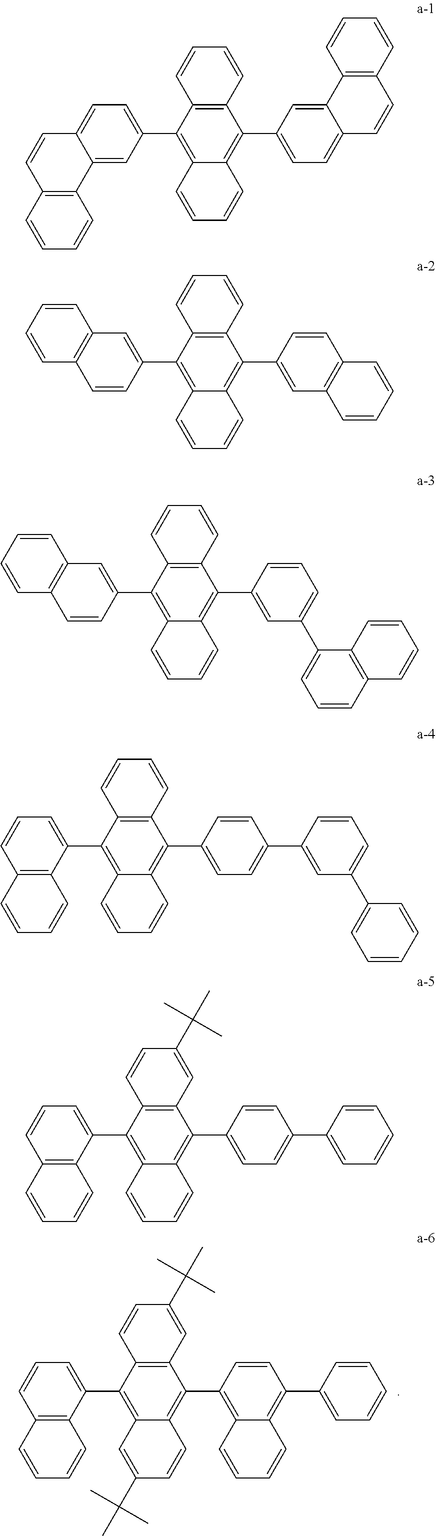

Non-limiting examples of the anthracene derivative used in the emission layer 110 of the organic EL device according to an embodiment of the present disclosure may include compounds represented by the following Formulae a-7 to a-12:

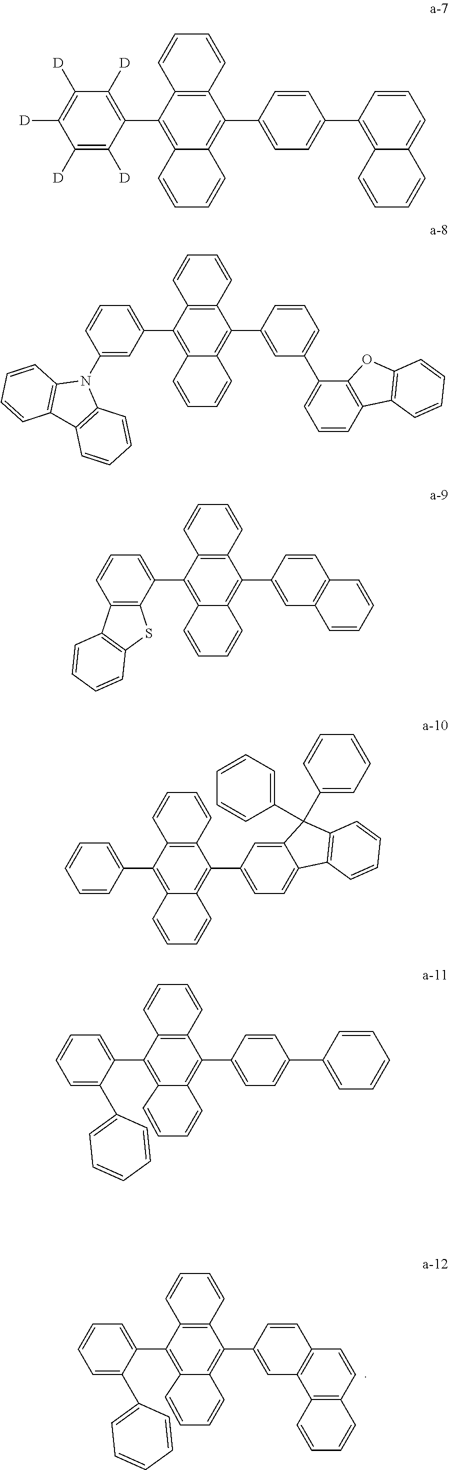

The emission layer 110 may include styryl derivatives (such as 1,4-bis[2-(3-N-ethylcarbazolyl)vinyl]benzene (BCzVB), 4-(di-p-tolylamino)-4′-[(di-p-tolylamino)styryl]stilbene (DPAVB), and N-(4-((E)-2-(6-((E)-4-(diphenylamino)styryl)naphthalen-2-yl)vinyl)phenyl-N-phenylbenzeneamine (N-BDAVBI)), perylene and derivatives thereof (such as 2,5,8,11-tetra-t-butylperylene (TBPe)), pyrene and derivatives thereof (such as 1,1-dipyrene, 1,4-dipyrenylbenzene, 1,4-bis(N,N-diphenylamino)pyrene), etc., without limitation, as a dopant.

The electron transport layer 112 may be on the emission layer 110 and may include, for example, tris(8-hydroxyquinolinolato)aluminum (Alq3) or a material having a nitrogen-containing aromatic ring (for example, a material including a pyridine ring such as 1,3,5-tri[(3-pyridyl)-phen-3-yl]benzene, a material including a triazine ring such as 2,4,6-tris(3′-(pyridine-3-yl)biphenyl-3-yl)1,3,5-triazine, and/or a material including an imidazole derivative such as 2-(4-N-phenylbenzoimidazolyl-1-ylphenyl)-9,10-dinaphthylanthracene).

The electron injection layer 114 may be formed on the electron transport layer 112 and may include, for example, lithium fluoride (LiF), lithium-8-quinolinato (LiQ), etc. The cathode 116 may be on the electron injection layer 114 and may be formed using a metal such as aluminum (Al), silver (Ag), lithium (Li), magnesium (Mg) and calcium (Ca) and/or a material such as ITO and IZO. Each layer may be formed by selecting an appropriate or suitable layer forming method depending on the material used such as a vacuum evaporation method, a sputtering method, and/or other suitable coating methods.

In the organic EL device 100 according to the present disclosure, a hole transport layer capable of realizing a decrease in driving voltage and increase in emission efficiency may be formed using the material for an organic EL device according to the present disclosure. The material for an organic EL device according to the present disclosure may be applied to an active matrix type (e.g., active matrix) organic EL display using thin film transistors (TFTs).

According to an embodiment of the present disclosure, the organic EL device 100 having a decreased driving voltage and high emission efficiency may be manufactured by using the material for an organic EL device according to the present disclosure in at least one stacking layer provided between the emission layer and the anode.

A method of synthesizing the material for an organic EL device and a method of manufacturing the organic EL device according to the present disclosure will be explained in more detail. However, the following examples are only for illustration, and the scope of the present disclosure is not limited thereto.

### EXAMPLES

### Preparation Method

### Synthesis of Compound 2

Compound 2 may be synthesized according to the following method. First, Compound A was synthesized as an intermediate.

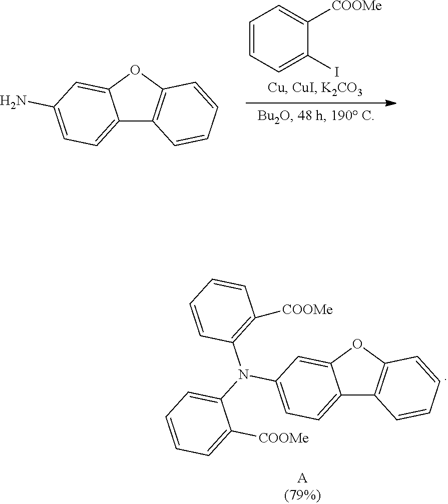 [SMILES: Nc1ccc2c(c1)oc1ccccc12 ; COC(=O)c1ccccc1I ; COC(=O)c1ccccc1N(c1ccc2c(c1)oc1ccccc12)c1ccccc1C(=O)OC]

Under an argon (Ar) atmosphere, 10.0 g of 3-aminodibenzofuran, 47.0 mL of methyl-2-iodobenzoate, 31.1 g of K₂CO₃ and 1.36 g of a copper powder were added to a 300 mL, three-necked flask, followed by heating and stirring in 100 mL of di-n-butyl ether as a solvent at about 190° C. for about 48 hours. After air cooling, water was added, the organic layer was separated, and the solvents were distilled. The crude product thus obtained was separated using silica gel column chromatography (using a mixed solvent of toluene and hexane) and recrystallized using a mixed solvent of toluene/ethanol to produce 19.5 g of Compound A as a white solid (Yield 79%). The molecular weight of Compound A measured by Fast Atom Bombardment-Mass Spectrometry (FAB-MS) was 452.

Compound B was synthesized using Compound A as a raw material according to the following method.

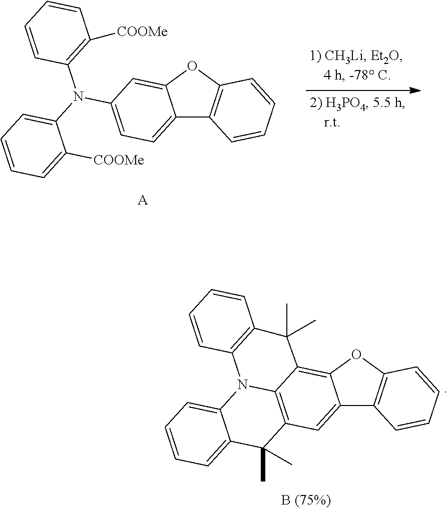

Under an argon atmosphere, 18.9 g of Compound A was added to a 300 mL, three-necked flask, followed by cooling in 80 mL of ether to −78° C. After that, 42 mL of an ether solution of 1 to 2 M of methyl lithium was added thereto, and the temperature was allowed to increase to room temperature, followed by reacting for about 4 hours. After the reaction, 60 mL of H₂SO₄ was slowly added in an ice bath. Dichloromethane was added, the organic layer was separated, and the solvents were distilled. The crude product thus obtained was dissolved in 85% aqueous H₃PO₄, followed by stirring at room temperature for about 2 hours. 2M NaOH aqueous solution was added, the reaction product was extracted with dichloromethane, and the solvents were distilled. The crude product thus obtained was separated using silica gel column chromatography (using a mixed solvent of dichloromethane and hexane) and recrystallized using a mixed solvent of toluene/hexane to produce 13.0 g of Compound B as a white solid (Yield 75%). The molecular weight of Compound B measured by FAB-MS was 416.

Compound C was synthesized using Compound B as a raw material according to the following method.

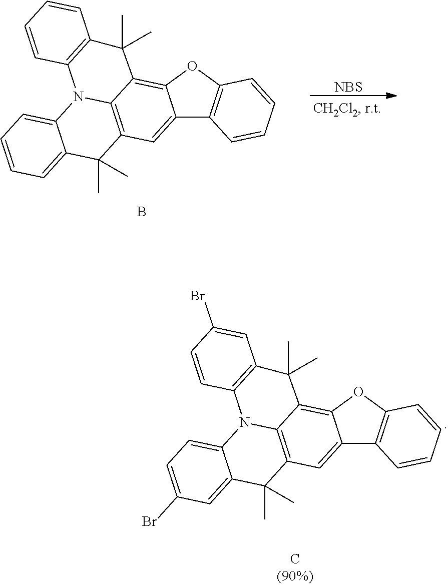

9.66 g of Compound B, 13.2 g of N-bromo succinimide (NBS) and 40 mL of dichloromethane were added to a 100 mL, two-necked flask followed by stirring at room temperature for 5 hours. Water was added to separate an organic layer, and the solvents were distilled. The crude product thus obtained was separated using silica gel column chromatography (using a mixed solvent of dichloromethane and hexane) and recrystallized using a mixed solvent of toluene/hexane to produce 9.94 g of Compound C as a white solid (Yield 90%). The molecular weight of Compound C measured by FAB-MS was 573.

Compound 2 as a final compound was synthesized using Compound C as a raw material according to the following method.

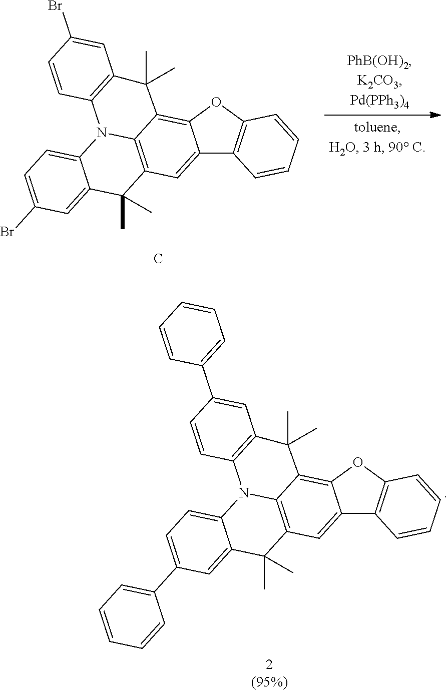

Under an argon atmosphere, 7.18 g of Compound C, 2.10 g of phenylboronic acid, 1.30 g of Pd(PPh₃)₄ and 4.21 g of potassium carbonate were added to a 300 mL, three-necked flask, followed by heating and stirring in a mixed solvent of 150 mL of toluene and 60 mL of water at about 90° C. for about 3 hours. After air cooling, water was added, an organic layer was separated, and solvents were distilled. The crude product thus obtained was separated by silica gel column chromatography (using a mixed solvent of dichloromethane and hexane) and recrystallized using a mixed solvent of toluene/hexane to produce 6.75 g of Compound 2 as a white solid (Yield 95%).

The molecular weight of Compound 2 measured by FAB-MS was 764. The chemical shift values (δ) of Compound 2 measured by ¹H-NMR (CDCl₃) were 7.89 (d, 2H, J=7.60 Hz), 7.66 (d, 1H, J=7.80 Hz), 7.63-7.60 (m, 2H), 7.55-7.50 (m, 8H), 7.41-7.32 (m, 6H), 6.93 (s, 1H), 6.61 (d, 2H, J=7.80 Hz), 1.73 (s, 6H), 1.66 (s, 6H).

### Synthesis of Compound 8

Compound 8 was synthesized according to substantially the same method used to synthesize Intermediate B in the method of Compound 2, except for changing methyl lithium to phenylmagnesium iodide. The molecular weight of Compound 8 measured by FAB-MS was 816. The chemical shift values (δ) of Compound 8 measured by ¹H-NMR (CDCl₃) were 7.91 (d, 1H, J=7.50 Hz), 7.66 (d, 1H, J=7.60 Hz), 7.53-7.50 (m, 10H), 7.37-7.26 (m, 17H), 7.16-7.10 (m, 8H), 6.85 (s, 1H), 6.57 (d, 2H, J=7.80 Hz), 1.73 (s, 6H), 1.66 (s, 6H).

### Synthesis of Compound 14

Compound 14 was synthesized according to substantially the same method used to synthesize Intermediate A in the method of Compound 2, except for changing 3-aminodibenzofuran to 3-aminodibenzothiophene. The molecular weight of Compound 14 measured by FAB-MS was 584. The chemical shift values (δ) of Compound 14 measured by ¹H-NMR (CDCl₃) were 7.79 (d, 2H, J=7.60 Hz), 7.76 (d, 1H, J=7.80 Hz), 7.63-7.60 (m, 2H), 7.54-7.50 (m, 8H), 7.39-7.30 (m, 6H), 6.83 (s, 1H), 6.55 (d, 2H, J=7.80 Hz), 1.71 (s, 6H), 1.66 (s, 6H).

Organic EL devices according to Examples 1 to 3 were manufactured using the following Compounds 2, 8 and 14 as hole transport materials by the above-described method:

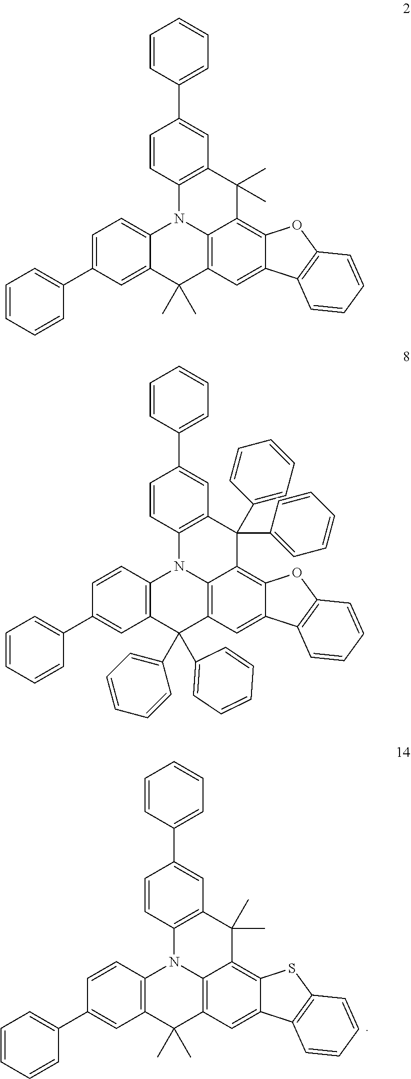 [SMILES: CC1(C)c2cc(-c3ccccc3)ccc2N2c3ccc(-c4ccccc4)cc3C(C)(C)c3c2c1cc1c3oc2ccccc21 ; c1ccc(-c2ccc3c(c2)C(c2ccccc2)(c2ccccc2)c2cc4c(oc5ccccc54)c4c2N3c2ccc(-c3ccccc3)cc2C4(c2ccccc2)c2ccccc2)cc1 ; CC1(C)c2cc(-c3ccccc3)ccc2N2c3ccc(-c4ccccc4)cc3C(C)(C)c3c2c1cc1c3sc2ccccc21]

For comparison, organic EL devices according to Comparative Examples 1 to 3 were manufactured using the following Compounds C-1 to C-3 as hole transport materials:

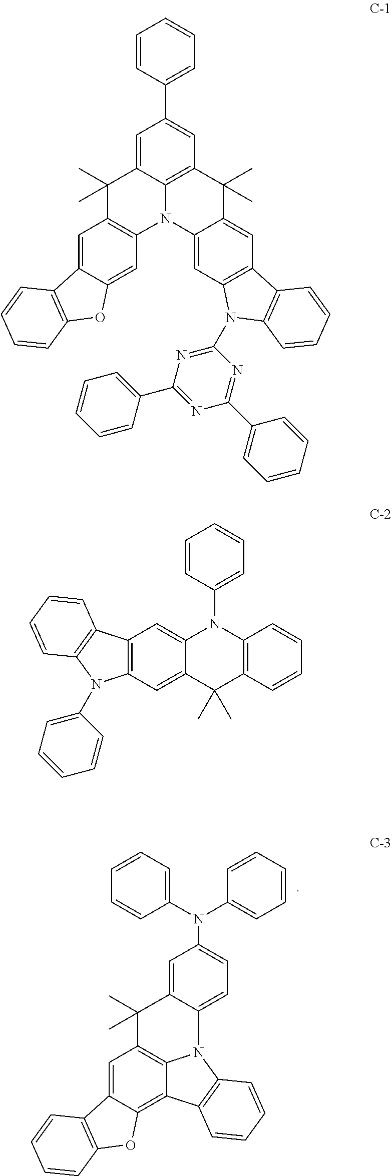 [SMILES: CC1(C)c2cc3c(cc2N2c4cc5c(cc4C(C)(C)c4cc(-c6ccccc6)cc1c42)c1ccccc1n5-c1nc(-c2ccccc2)nc(-c2ccccc2)n1)oc1ccccc13 ; CC1(C)c2ccccc2N(c2ccccc2)c2cc3c4ccccc4n(-c4ccccc4)c3cc21 ; CC1(C)c2cc(N(c3ccccc3)c3ccccc3)ccc2-n2c3ccccc3c3c4oc5ccccc5c4cc1c32]

In an embodiment of the present disclosure, a transparent glass substrate was used as a substrate 102, an anode 104 was formed using ITO to a layer thickness of about 150 nm, a hole injection layer 106 was formed using 2-TNATA to a layer thickness of about 60 nm, a hole transport layer 108 was formed using each compound according to the Examples and Comparative Examples to a layer thickness of about 70 nm, an emission layer 110 was formed using ADN doped with 5% TBP to a layer thickness of about 25 nm, an electron transport layer 112 was formed using Alq3 to a layer thickness of about 25 nm, an electron injection layer 114 was formed using LiF to a layer thickness of about 1 nm, and a cathode 116 was formed using Al to a layer thickness of about 100 nm.

The voltages and emission efficiencies of the organic EL devices according to Examples 1 to 3 and Comparative Examples 1 to 3 were evaluated at a current density of 10 mA/cm².

TABLE 1

| | | Driving | Emission |
|---|---|---|---|
| | | voltage | efficiency |
| | Hole transport layer | (V) | (cd/A) |
| Example 1 | Example Compound 2 | 5.6 | 6.7 |
| Example 2 | Example Compound 8 | 5.6 | 6.7 |
| Example 3 | Example Compound 14 | 6.0 | 6.4 |
| Comparative | Comparative Compound | 6.3 | 5.2 |
| Example 1 | C-1 | | |
| Comparative | Comparative Compound | 6.5 | 5.0 |
| Example 2 | C-2 | | |
| Comparative | Comparative Compound | 6.6 | 5.2 |
| Example 3 | C-3 | | |

From the results in Table 1, organic EL devices using the materials for an organic EL device according to Examples 1 to 3 (e.g., benzofuroacridine-like derivatives) in the hole transport layers had lower driving voltages and higher emission efficiencies compared to the compounds of Comparative Examples 1 to 3. It is thought that the materials for an organic EL device according to Examples 1 to 3 are plane-immobilized due to the crosslinking structure using quaternary carbon, and owing to the crosslinking structure, the HOMO-LUMO gap may be increased and energy transfer may be restrained or reduced, thereby increasing the emission efficiency of the organic EL device.

In Comparative Example 1, the compound included a carbazole part, a dibenzofuran part and a triazine part, such that its electron accepting properties may have been increased and its hole transport properties may have been negatively affected, thereby inducing low efficiency. In Comparative Example 3, the compound included an indolo[3,2,1-de]acridine part, and the LUMO energy level may have been largely deteriorated (e.g., lowered, resulting in a smaller HOMO-LUMO gap), thereby inducing a lowered efficiency.

The material for an organic EL device according to the present disclosure may be plane-immobilized by quaternary carbon or silicon, the HOMO-LUMO gap may be increased, and energy transfer may be restrained or reduced, thereby enabling high emission efficiency in the organic EL device. In some embodiments, remarkable effects may be obtained in the blue emission region.

One or more embodiments of the present disclosure provide a material for an organic EL device having high emission efficiency with a low driving voltage and an organic EL device including the same. One or more embodiments of the present disclosure provide a material for an organic EL device realizing high emission efficiency and a long driving voltage in the blue and/or green emission regions, and an organic EL device including the same in at least one stacking layer between an anode and an emission layer. According to an embodiment of the present disclosure, the material may be plane-immobilized by making a cross-linking structure via quaternary carbon or silicon. According to the crosslinking structure, the HOMO-LUMO gap may be enlarged and energy transfer may be restrained or reduced, thereby realizing high emission efficiency in the organic EL device.

While one or more example embodiments have been described with reference to the drawings, it will be understood by those of ordinary skill in the art that various changes in form and details may be made therein without departing from the spirit and scope of the present disclosure as defined by the following claims and equivalents thereof.

As used herein, expressions such as “at least one of”, “one of”, “at least one selected from”, and “one selected from”, when preceding a list of elements, modify the entire list of elements and do not modify the individual elements of the list. Further, the use of “may” when describing embodiments of the present disclosure refers to “one or more embodiments of the present disclosure”.

In addition, as used herein, the terms “use”, “using”, and “used” may be considered synonymous with the terms “utilize”, “utilizing”, and “utilized”, respectively.

As used herein, the terms “substantially”, “about”, and similar terms are used as terms of approximation and not as terms of degree, and are intended to account for the inherent deviations in measured or calculated values that would be recognized by those of ordinary skill in the art.

Also, any numerical range recited herein is intended to include all subranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

The above-disclosed subject matter is to be considered illustrative and not restrictive, and the appended claims are intended to cover all such modifications, enhancements, and other embodiments, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

What is claimed is:

1. A material for an organic electroluminescent (EL) device represented by Formula 1: 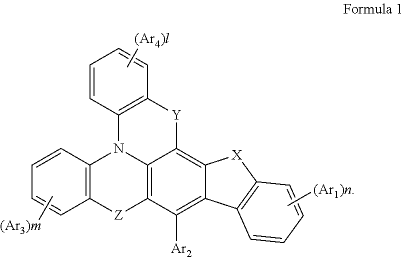
wherein, in Formula 1,
X is selected from O, N and S,
Y and Z are each independently selected from CAr₅Ar₆ and SiAr₇Ar₈,
Ar₁ to Ar₈ are each independently selected from a substituted or unsubstituted alkyl group having 1 to 30 carbon atoms, a substituted or unsubstituted aryl group having 6 to 30 carbon atoms for forming a ring, a substituted or unsubstituted heteroaryl group having 5 to 30 carbon atoms for forming a ring, a silyl group, a halogen atom, deuterium, and hydrogen, and
l to n are each independently selected from an integer from 0 to 4.

2. The material for an organic EL device of claim 1, wherein X is O, and Y and Z are each independently CAr₅Ar₆ in Formula 1.

3. The material for an organic EL device of claim 1, wherein Ar₁ to Ar₈ are each independently selected from a phenyl group, a biphenyl group, a naphthyl group, a phenylnaphthyl group, a naphthylphenyl group, and hydrogen.

4. The material for an organic EL device of claim 1, wherein the material represented by Formula 1 is represented by one selected from Formulae 2 to 5: 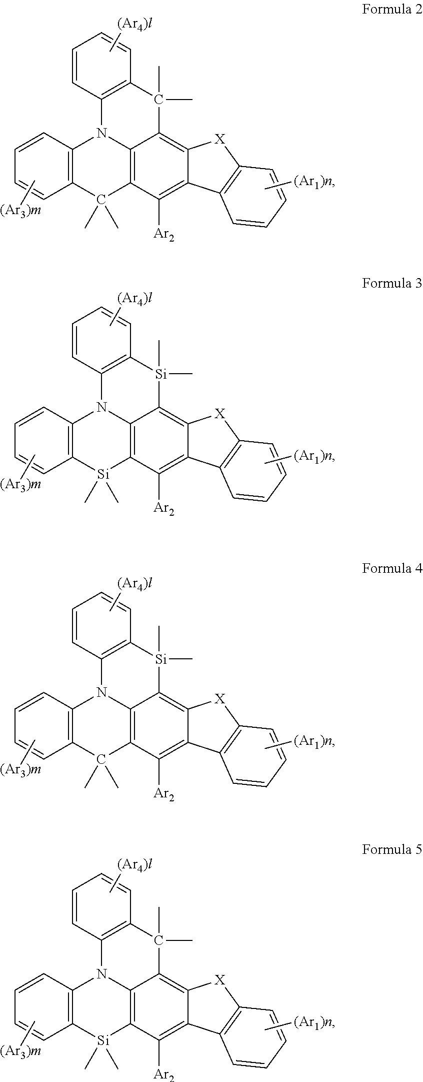
wherein in Formulae 2 to 5,
the definitions of X, Ar₁ to Ar₄ and l to n are the same as the definitions of X, Ar₁ to Ar₄, and l to n in Formula 1.

5. The material for an organic EL device of claim 1, wherein the material represented by Formula 1 is selected from Compounds 1 to 7:

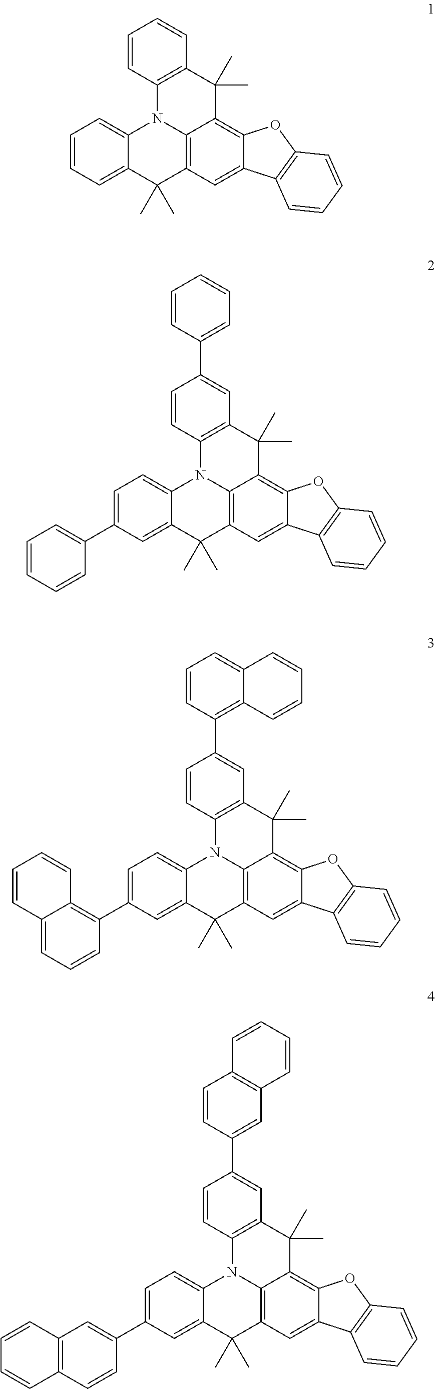 [SMILES: CC1(C)c2ccccc2N2c3ccccc3C(C)(C)c3c2c1cc1c3oc2ccccc21 ; CC1(C)c2cc(-c3ccccc3)ccc2N2c3ccc(-c4ccccc4)cc3C(C)(C)c3c2c1cc1c3oc2ccccc21 ; CC1(C)c2cc(-c3cccc4ccccc34)ccc2N2c3ccc(-c4cccc5ccccc45)cc3C(C)(C)c3c2c1cc1c3oc2ccccc21 ; CC1(C)c2cc(-c3ccc4ccccc4c3)ccc2N2c3ccc(-c4ccc5ccccc5c4)cc3C(C)(C)c3c2c1cc1c3oc2ccccc21]

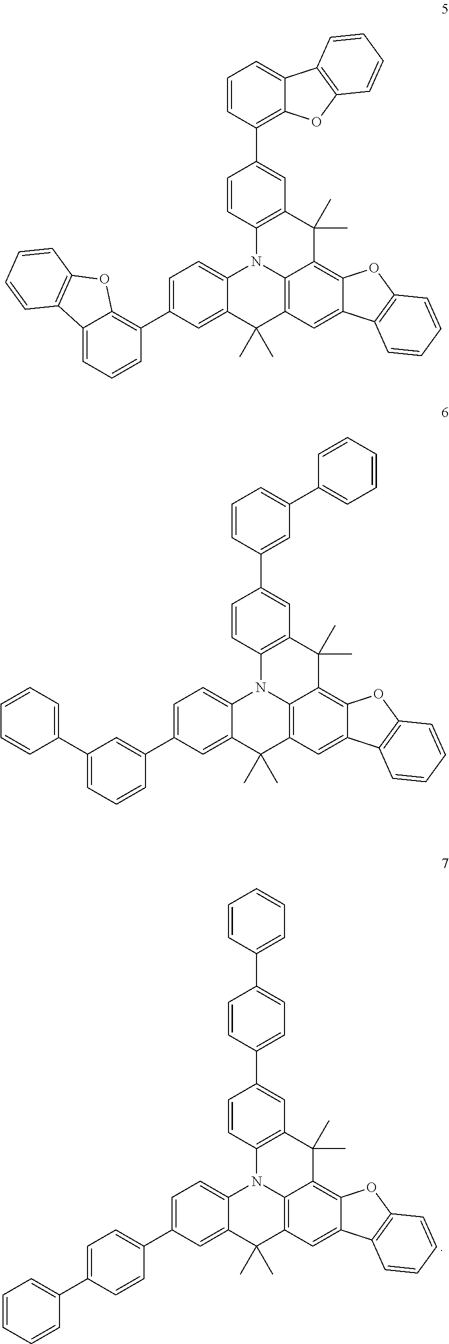 [SMILES: CC1(C)c2cc(-c3cccc4c3oc3ccccc34)ccc2N2c3ccc(-c4cccc5c4oc4ccccc45)cc3C(C)(C)c3c2c1cc1c3oc2ccccc21 ; CC1(C)c2cc(-c3cccc(-c4ccccc4)c3)ccc2N2c3ccc(-c4cccc(-c5ccccc5)c4)cc3C(C)(C)c3c2c1cc1c3oc2ccccc21 ; CC1(C)c2cc(-c3ccc(-c4ccccc4)cc3)ccc2N2c3ccc(-c4ccc(-c5ccccc5)cc4)cc3C(C)(C)c3c2c1cc1c3oc2ccccc21]

6. The material for an organic EL device of claim 1, wherein the material represented by Formula 1 is selected from Compounds 8 to 14:

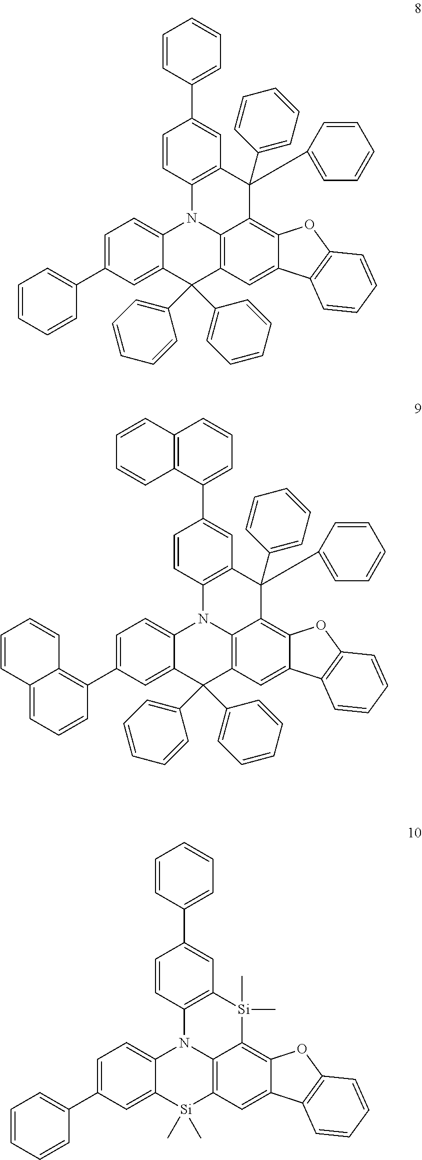 [SMILES: c1ccc(-c2ccc3c(c2)C(c2ccccc2)(c2ccccc2)c2cc4c(oc5ccccc54)c4c2N3c2ccc(-c3ccccc3)cc2C4(c2ccccc2)c2ccccc2)cc1 ; c1ccc(C2(c3ccccc3)c3cc(-c4cccc5ccccc45)ccc3N3c4ccc(-c5cccc6ccccc56)cc4C(c4ccccc4)(c4ccccc4)c4c3c2cc2c4oc3ccccc32)cc1 ; C[Si]1(C)c2cc(-c3ccccc3)ccc2N2c3ccc(-c4ccccc4)cc3[Si](C)(C)c3c2c1cc1c3oc2ccccc21]

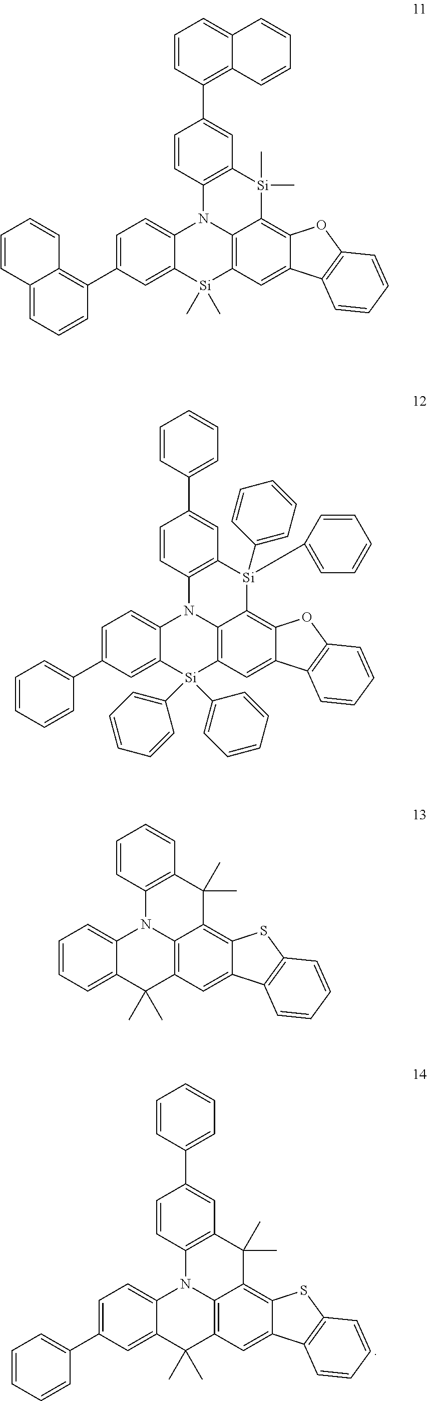 [SMILES: C[Si]1(C)c2cc(-c3cccc4ccccc34)ccc2N2c3ccc(-c4cccc5ccccc45)cc3[Si](C)(C)c3c2c1cc1c3oc2ccccc21 ; c1ccc(-c2ccc3c(c2)[Si](c2ccccc2)(c2ccccc2)c2cc4c(oc5ccccc54)c4c2N3c2ccc(-c3ccccc3)cc2[Si]4(c2ccccc2)c2ccccc2)cc1 ; CC1(C)c2ccccc2N2c3ccccc3C(C)(C)c3c2c1cc1c3sc2ccccc21 ; CC1(C)c2cc(-c3ccccc3)ccc2N2c3ccc(-c4ccccc4)cc3C(C)(C)c3c2c1cc1c3sc2ccccc21]

7. The material for an organic EL device of claim 1, wherein the material represented by Formula 1 is selected from Compounds 15 to 20:

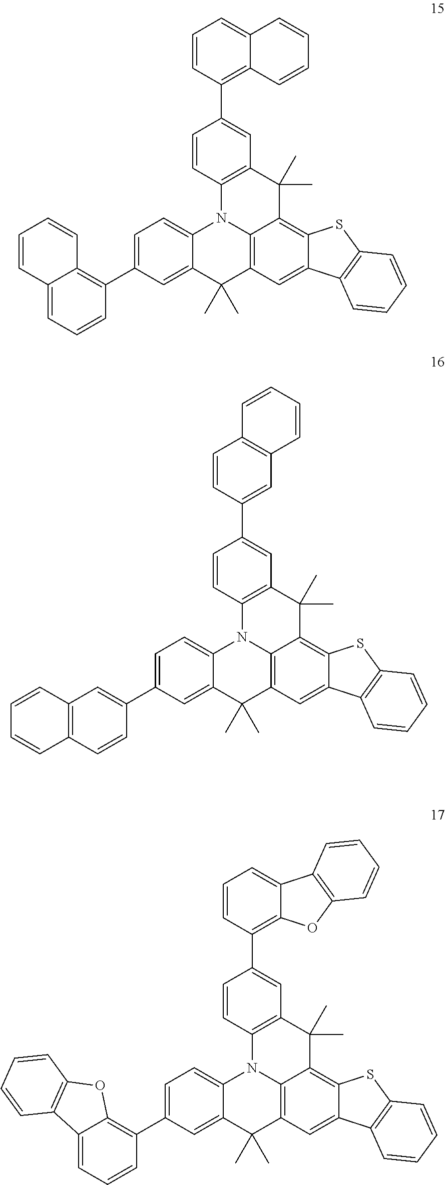 [SMILES: CC1(C)c2cc(-c3cccc4ccccc34)ccc2N2c3ccc(-c4cccc5ccccc45)cc3C(C)(C)c3c2c1cc1c3sc2ccccc21 ; CC1(C)c2cc(-c3ccc4ccccc4c3)ccc2N2c3ccc(-c4ccc5ccccc5c4)cc3C(C)(C)c3c2c1cc1c3sc2ccccc21 ; CC1(C)c2cc(-c3cccc4c3oc3ccccc34)ccc2N2c3ccc(-c4cccc5c4oc4ccccc45)cc3C(C)(C)c3c2c1cc1c3sc2ccccc21]

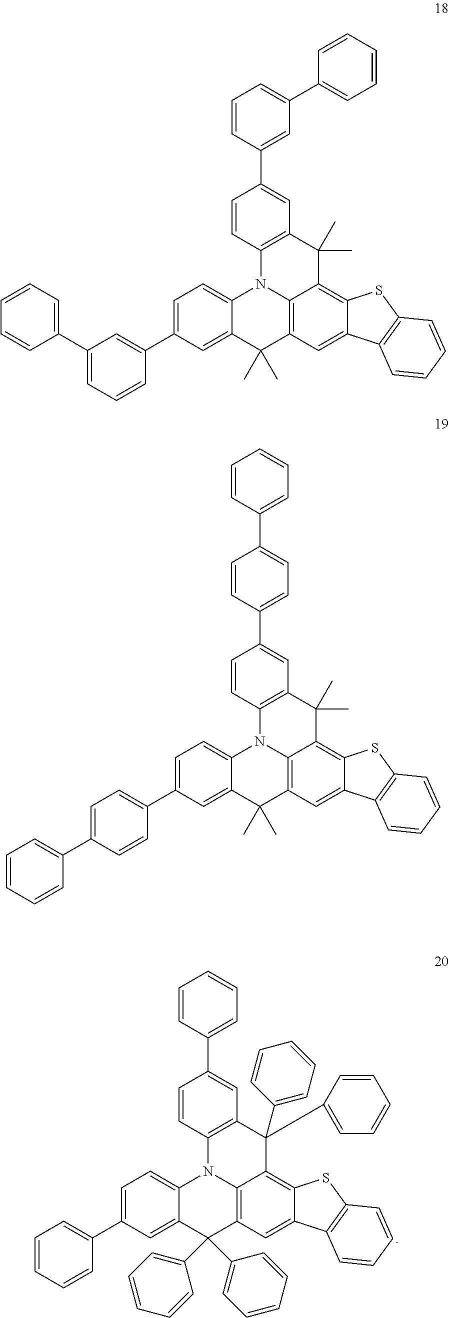 [SMILES: CC1(C)c2cc(-c3cccc(-c4ccccc4)c3)ccc2N2c3ccc(-c4cccc(-c5ccccc5)c4)cc3C(C)(C)c3c2c1cc1c3sc2ccccc21 ; CC1(C)c2cc(-c3ccc(-c4ccccc4)cc3)ccc2N2c3ccc(-c4ccc(-c5ccccc5)cc4)cc3C(C)(C)c3c2c1cc1c3sc2ccccc21 ; c1ccc(-c2ccc3c(c2)C(c2ccccc2)(c2ccccc2)c2cc4c(sc5ccccc54)c4c2N3c2ccc(-c3ccccc3)cc2C4(c2ccccc2)c2ccccc2)cc1]

8. The material for an organic EL device of claim 1, wherein the material represented by Formula 1 is selected from Compounds 21 to 27:

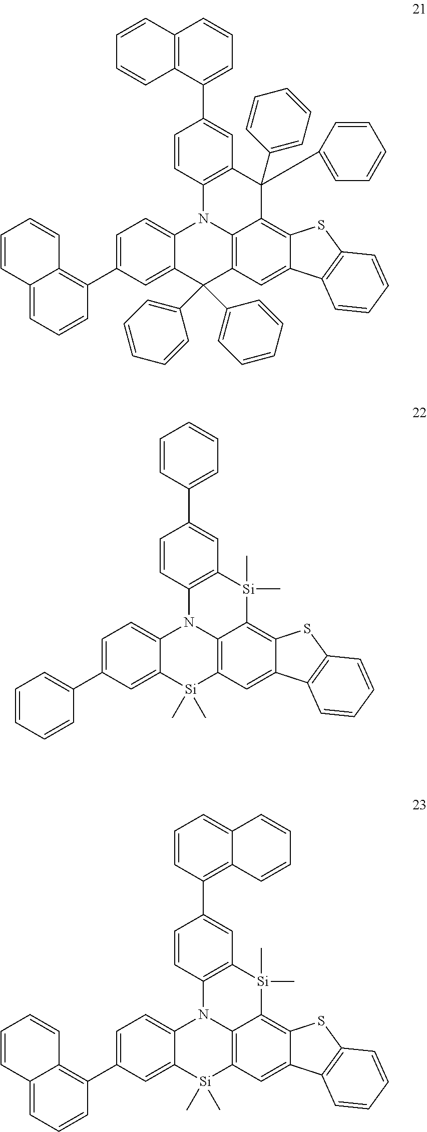 [SMILES: c1ccc(C2(c3ccccc3)c3cc(-c4cccc5ccccc45)ccc3N3c4ccc(-c5cccc6ccccc56)cc4C(c4ccccc4)(c4ccccc4)c4c3c2cc2c4sc3ccccc32)cc1 ; C[Si]1(C)c2cc(-c3ccccc3)ccc2N2c3ccc(-c4ccccc4)cc3[Si](C)(C)c3c2c1cc1c3sc2ccccc21 ; C[Si]1(C)c2cc(-c3cccc4ccccc34)ccc2N2c3ccc(-c4cccc5ccccc45)cc3[Si](C)(C)c3c2c1cc1c3sc2ccccc21]

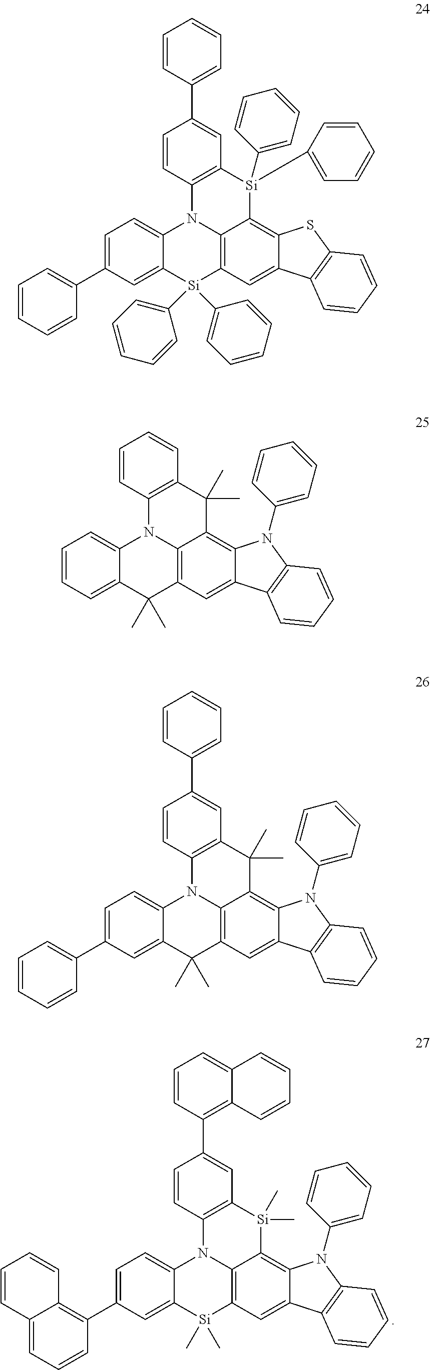 [SMILES: c1ccc(-c2ccc3c(c2)[Si](c2ccccc2)(c2ccccc2)c2cc4c(sc5ccccc54)c4c2N3c2ccc(-c3ccccc3)cc2[Si]4(c2ccccc2)c2ccccc2)cc1 ; CC1(C)c2ccccc2N2c3ccccc3C(C)(C)c3c2c1cc1c2ccccc2n(-c2ccccc2)c31 ; CC1(C)c2cc(-c3ccccc3)ccc2N2c3ccc(-c4ccccc4)cc3C(C)(C)c3c2c1cc1c2ccccc2n(-c2ccccc2)c31 ; C[Si]1(C)c2cc(-c3cccc4ccccc34)ccc2N2c3ccc(-c4cccc5ccccc45)cc3[Si](C)(C)c3c2c1cc1c2ccccc2n(-c2ccccc2)c31]

9. The material for an organic EL device of claim 1, wherein the material represented by Formula 1 selected from Compounds 28 to 33:

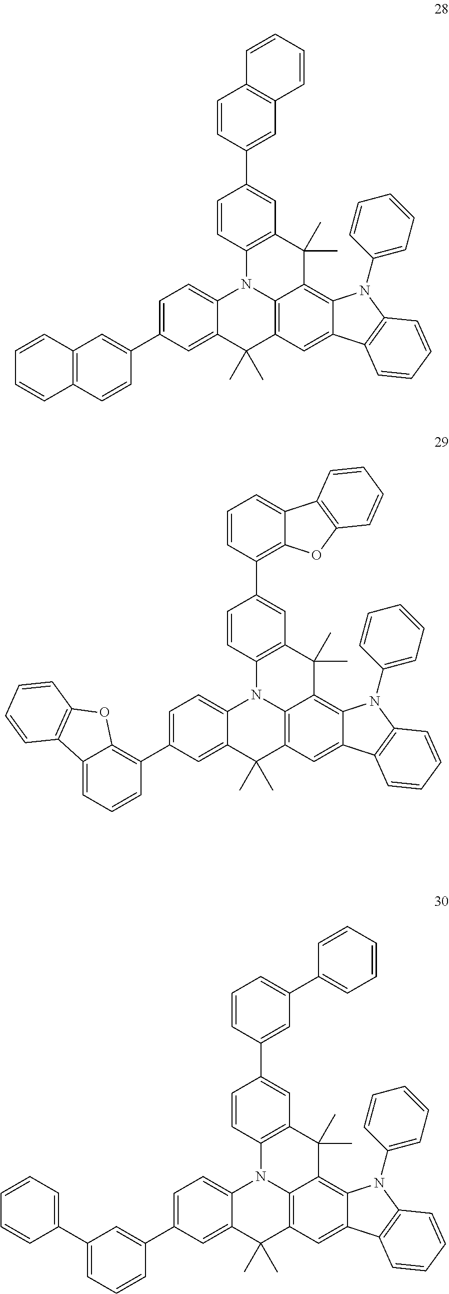 [SMILES: CC1(C)c2cc(-c3ccc4ccccc4c3)ccc2N2c3ccc(-c4ccc5ccccc5c4)cc3C(C)(C)c3c2c1cc1c2ccccc2n(-c2ccccc2)c31 ; CC1(C)c2cc(-c3cccc4c3oc3ccccc34)ccc2N2c3ccc(-c4cccc5c4oc4ccccc45)cc3C(C)(C)c3c2c1cc1c2ccccc2n(-c2ccccc2)c31 ; CC1(C)c2cc(-c3cccc(-c4ccccc4)c3)ccc2N2c3ccc(-c4cccc(-c5ccccc5)c4)cc3C(C)(C)c3c2c1cc1c2ccccc2n(-c2ccccc2)c31]

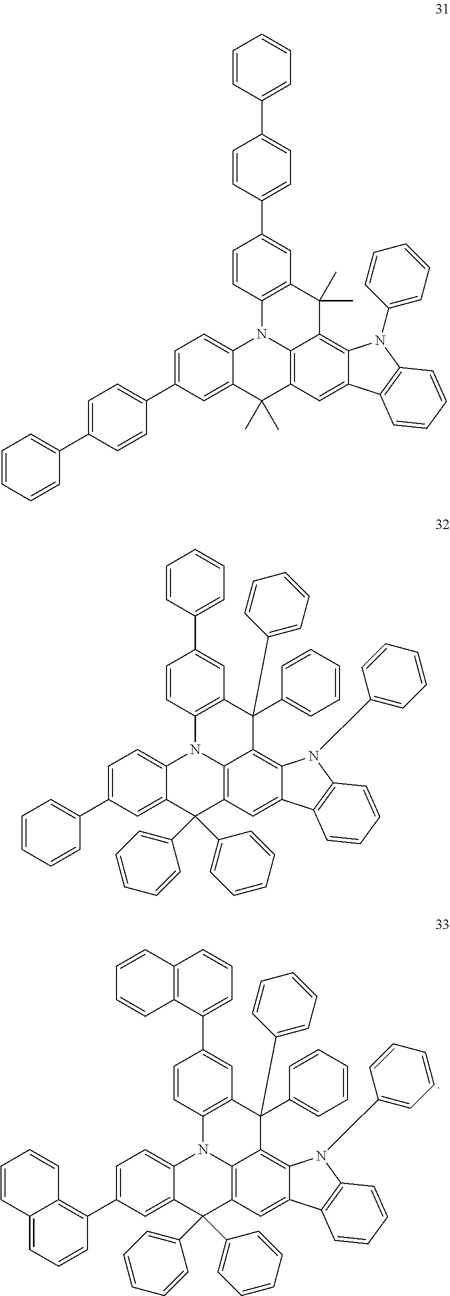 [SMILES: CC1(C)c2cc(-c3ccc(-c4ccccc4)cc3)ccc2N2c3ccc(-c4ccc(-c5ccccc5)cc4)cc3C(C)(C)c3c2c1cc1c2ccccc2n(-c2ccccc2)c31 ; c1ccc(-c2ccc3c(c2)C(c2ccccc2)(c2ccccc2)c2cc4c5ccccc5n(-c5ccccc5)c4c4c2N3c2ccc(-c3ccccc3)cc2C4(c2ccccc2)c2ccccc2)cc1 ; c1ccc(-n2c3ccccc3c3cc4c5c(c32)C(c2ccccc2)(c2ccccc2)c2cc(-c3cccc6ccccc36)ccc2N5c2ccc(-c3cccc5ccccc35)cc2C4(c2ccccc2)c2ccccc2)cc1]

10. The material for an organic EL device of claim 1, wherein the material represented by Formula 1 is selected from Compounds 34 to 39:

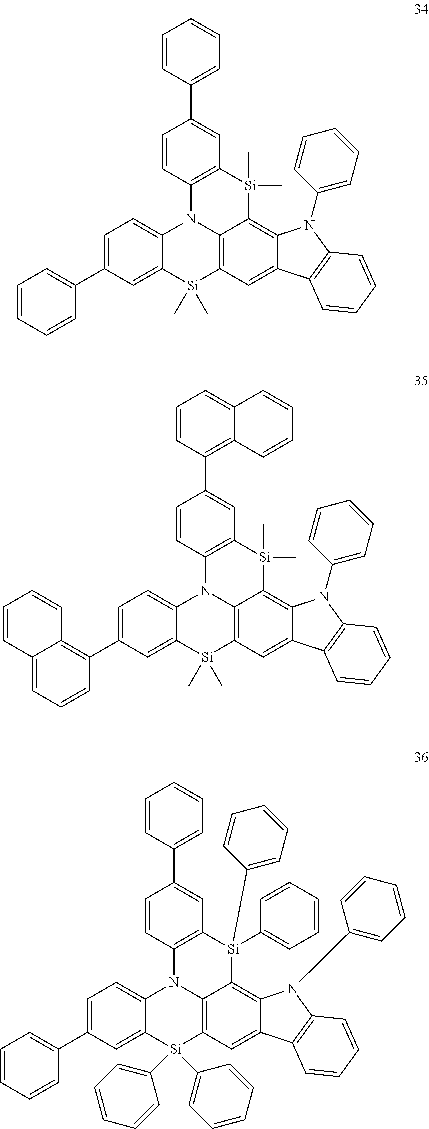 [SMILES: C[Si]1(C)c2cc(-c3ccccc3)ccc2N2c3ccc(-c4ccccc4)cc3[Si](C)(C)c3c2c1cc1c2ccccc2n(-c2ccccc2)c31 ; C[Si]1(C)c2cc(-c3cccc4ccccc34)ccc2N2c3ccc(-c4cccc5ccccc45)cc3[Si](C)(C)c3c2c1cc1c2ccccc2n(-c2ccccc2)c31 ; c1ccc(-c2ccc3c(c2)[Si](c2ccccc2)(c2ccccc2)c2cc4c5ccccc5n(-c5ccccc5)c4c4c2N3c2ccc(-c3ccccc3)cc2[Si]4(c2ccccc2)c2ccccc2)cc1]

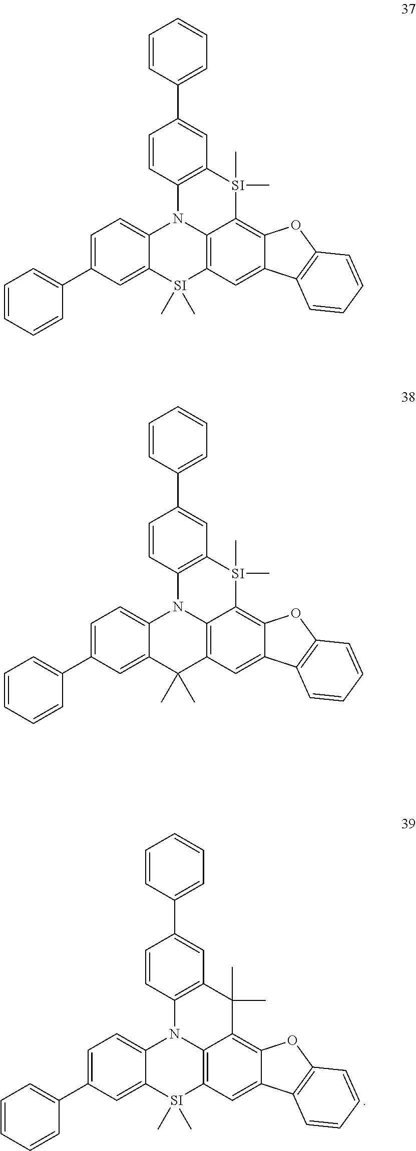

11. An organic electroluminescent (EL) device comprising a material for an organic EL device represented by Formula 1, the material represented by Formula 1 being included in at least one stacking layer between an emission layer and an anode: 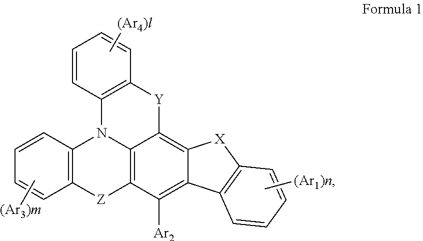
wherein in Formula 1,
X is selected from O, N and S,
Y and Z are each independently selected from CAr₅Ar₆ and SiAr₇Ar₈,
Ar₁ to Ar₈ are each independently selected from a substituted or unsubstituted alkyl group having 1 to 30 carbon atoms, a substituted or unsubstituted aryl group having 6 to 30 carbon atoms for forming a ring, a substituted or unsubstituted heteroaryl group having 5 to 30 carbon atoms for forming a ring, a silyl group, a halogen atom, deuterium, and hydrogen, and
l to n are each independently an integer selected from 0 to 4.

12. The organic EL device of claim 11, wherein X is O, and Y and Z are CAr₅Ar₆ in Formula 1.

13. The organic EL device of claim 11, wherein Ar₁ to Ar₈ are each independently selected from a phenyl group, a biphenyl group, a naphthyl group, a phenylnaphthyl group, a naphthylphenyl group, and hydrogen.

14. The organic EL device of claim 11, wherein the material represented by Formula 1 is represented by one selected from Formulae 2 to 5: 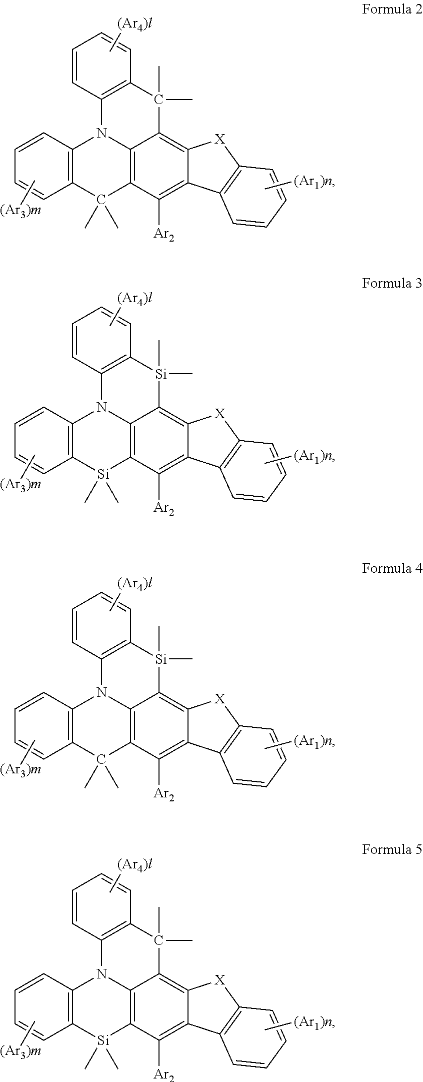
wherein in Formulae 2 to 5,
the definitions of X, Ar₁ to Ar₄ and l to n are the same as the definitions of X, Ar₁ to Ar₄ and l to n in Formula 1.

15. The organic EL device of claim 11, wherein the material represented by Formula 1 is represented by one selected from Compounds 1 to 7:

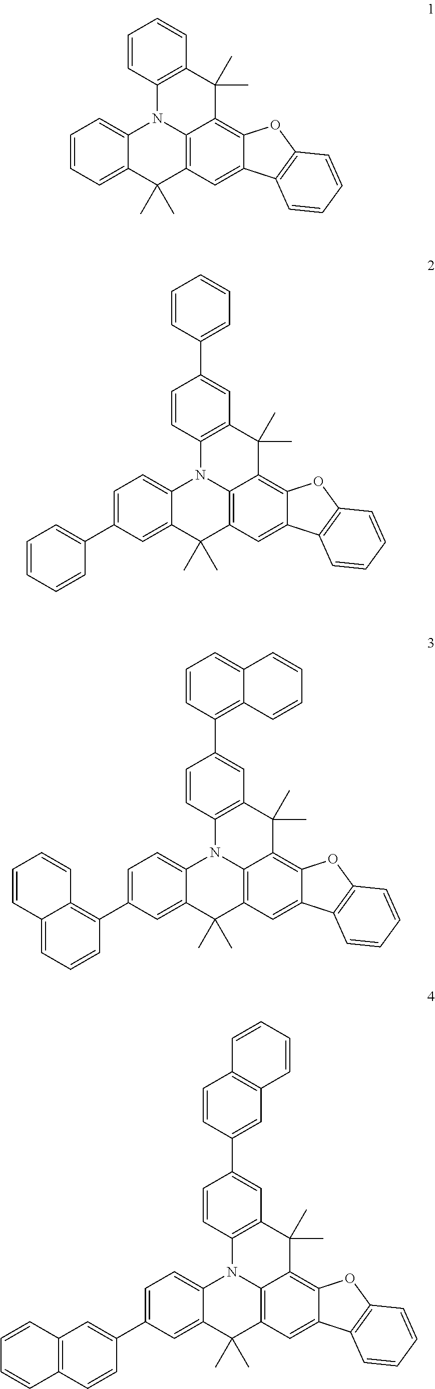 [SMILES: CC1(C)c2ccccc2N2c3ccccc3C(C)(C)c3c2c1cc1c3oc2ccccc21 ; CC1(C)c2cc(-c3ccccc3)ccc2N2c3ccc(-c4ccccc4)cc3C(C)(C)c3c2c1cc1c3oc2ccccc21 ; CC1(C)c2cc(-c3cccc4ccccc34)ccc2N2c3ccc(-c4cccc5ccccc45)cc3C(C)(C)c3c2c1cc1c3oc2ccccc21 ; CC1(C)c2cc(-c3ccc4ccccc4c3)ccc2N2c3ccc(-c4ccc5ccccc5c4)cc3C(C)(C)c3c2c1cc1c3oc2ccccc21]

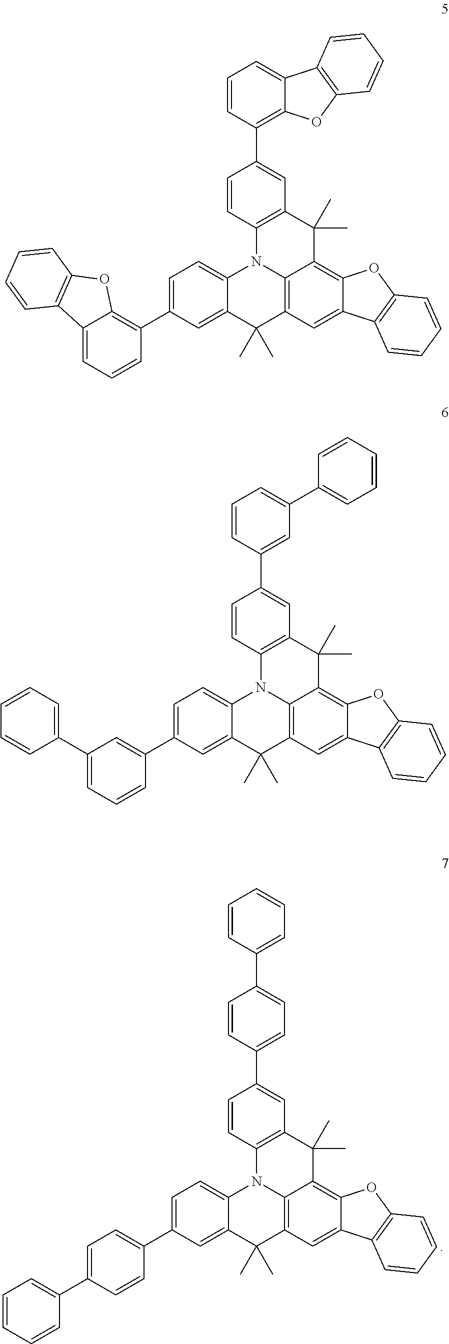 [SMILES: CC1(C)c2cc(-c3cccc4c3oc3ccccc34)ccc2N2c3ccc(-c4cccc5c4oc4ccccc45)cc3C(C)(C)c3c2c1cc1c3oc2ccccc21 ; CC1(C)c2cc(-c3cccc(-c4ccccc4)c3)ccc2N2c3ccc(-c4cccc(-c5ccccc5)c4)cc3C(C)(C)c3c2c1cc1c3oc2ccccc21 ; CC1(C)c2cc(-c3ccc(-c4ccccc4)cc3)ccc2N2c3ccc(-c4ccc(-c5ccccc5)cc4)cc3C(C)(C)c3c2c1cc1c3oc2ccccc21]

16. The organic EL device of claim 11, wherein the material represented by Formula 1 is represented by one selected from Compounds 8 to 14:

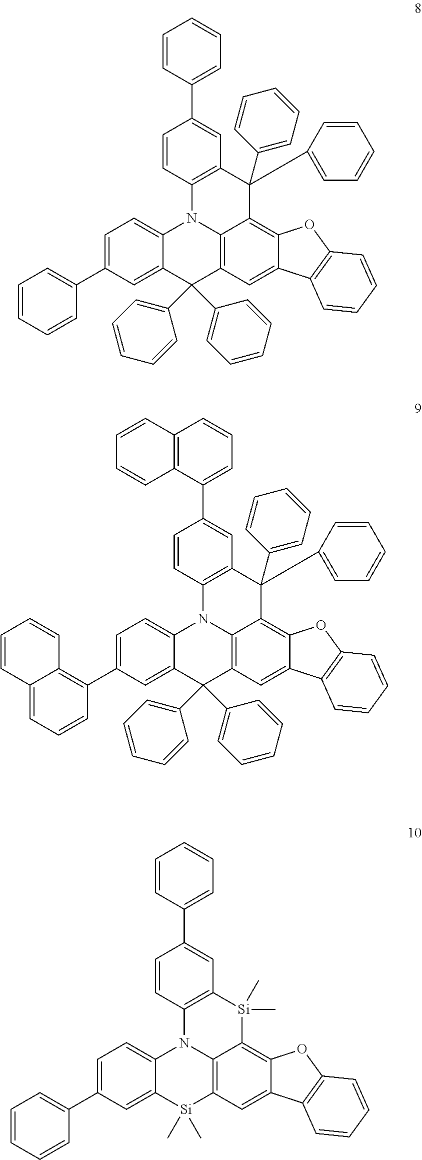 [SMILES: c1ccc(-c2ccc3c(c2)C(c2ccccc2)(c2ccccc2)c2cc4c(oc5ccccc54)c4c2N3c2ccc(-c3ccccc3)cc2C4(c2ccccc2)c2ccccc2)cc1 ; c1ccc(C2(c3ccccc3)c3cc(-c4cccc5ccccc45)ccc3N3c4ccc(-c5cccc6ccccc56)cc4C(c4ccccc4)(c4ccccc4)c4c3c2cc2c4oc3ccccc32)cc1 ; C[Si]1(C)c2cc(-c3ccccc3)ccc2N2c3ccc(-c4ccccc4)cc3[Si](C)(C)c3c2c1cc1c3oc2ccccc21]

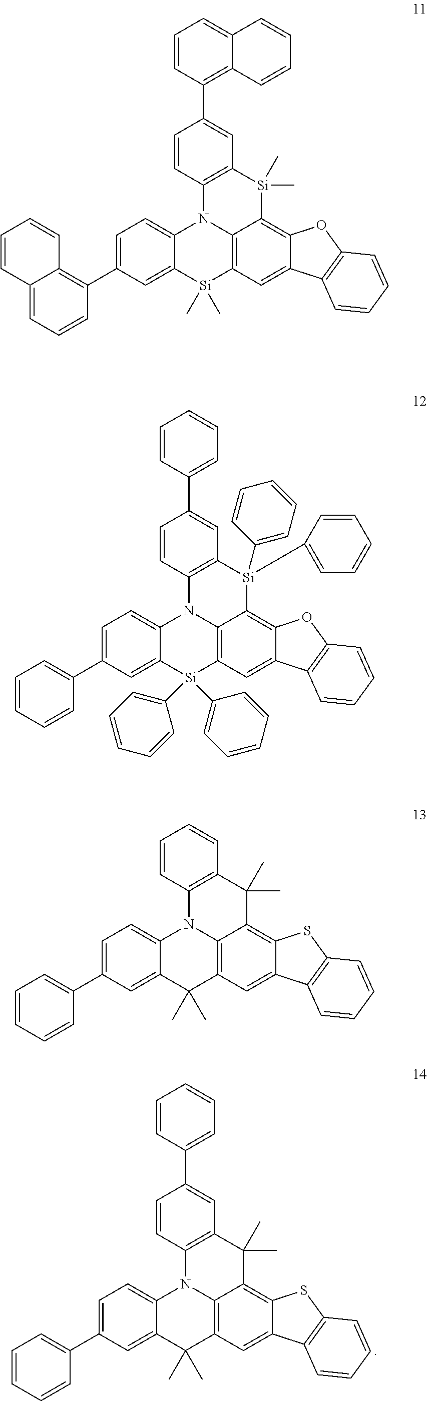 [SMILES: C[Si]1(C)c2cc(-c3cccc4ccccc34)ccc2N2c3ccc(-c4cccc5ccccc45)cc3[Si](C)(C)c3c2c1cc1c3oc2ccccc21 ; c1ccc(-c2ccc3c(c2)[Si](c2ccccc2)(c2ccccc2)c2cc4c(oc5ccccc54)c4c2N3c2ccc(-c3ccccc3)cc2[Si]4(c2ccccc2)c2ccccc2)cc1 ; CC1(C)c2cc(-c3ccccc3)ccc2N2c3ccccc3C(C)(C)c3c2c1cc1c3sc2ccccc21 ; CC1(C)c2cc(-c3ccccc3)ccc2N2c3ccc(-c4ccccc4)cc3C(C)(C)c3c2c1cc1c3sc2ccccc21]

17. The organic EL device of claim 11, wherein the material represented by Formula 1 is represented by one selected from Compounds 15 to 20:

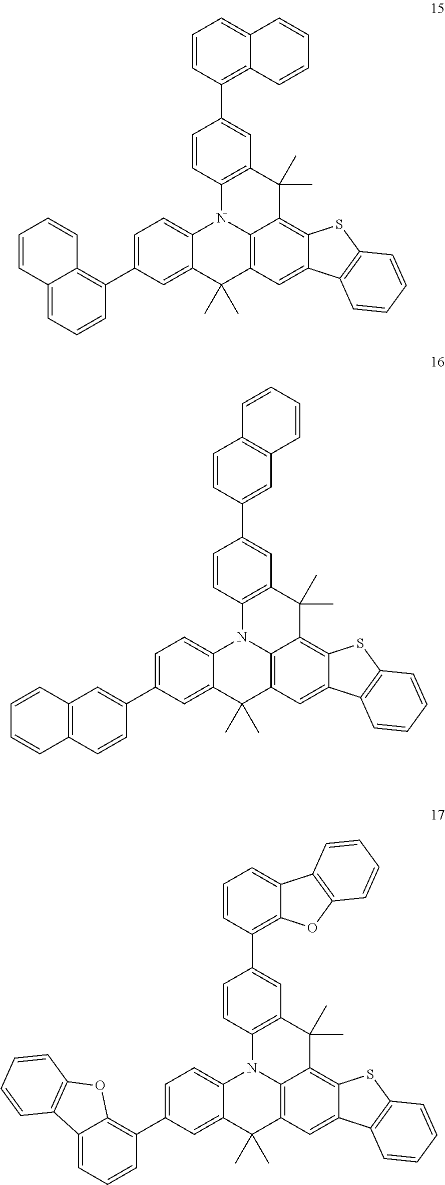 [SMILES: CC1(C)c2cc(-c3cccc4ccccc34)ccc2N2c3ccc(-c4cccc5ccccc45)cc3C(C)(C)c3c2c1cc1c3sc2ccccc21 ; CC1(C)c2cc(-c3ccc4ccccc4c3)ccc2N2c3ccc(-c4ccc5ccccc5c4)cc3C(C)(C)c3c2c1cc1c3sc2ccccc21 ; CC1(C)c2cc(-c3cccc4c3oc3ccccc34)ccc2N2c3ccc(-c4cccc5c4oc4ccccc45)cc3C(C)(C)c3c2c1cc1c3sc2ccccc21]

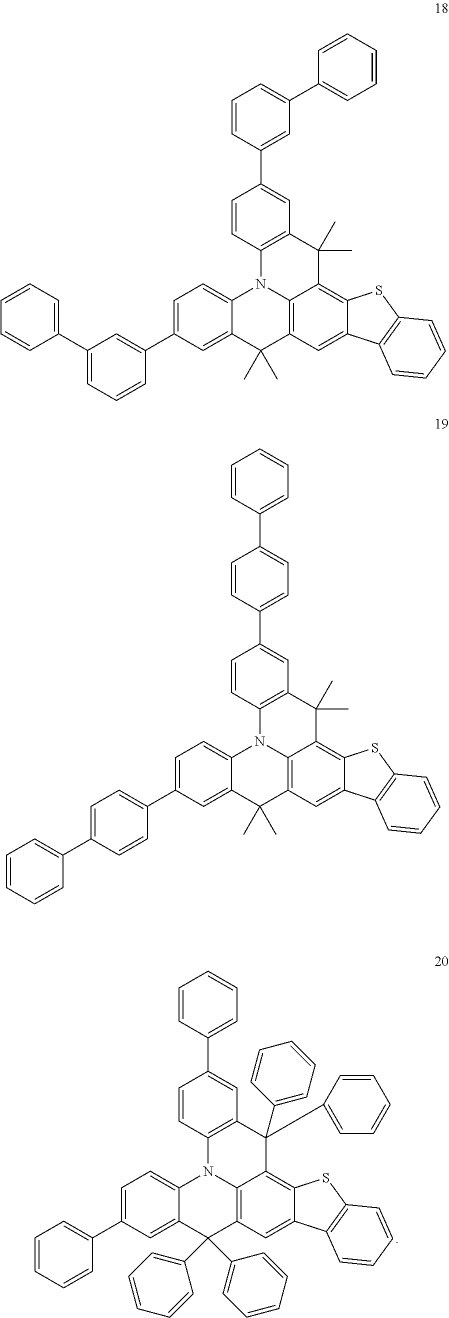 [SMILES: CC1(C)c2cc(-c3cccc(-c4ccccc4)c3)ccc2N2c3ccc(-c4cccc(-c5ccccc5)c4)cc3C(C)(C)c3c2c1cc1c3sc2ccccc21 ; CC1(C)c2cc(-c3ccc(-c4ccccc4)cc3)ccc2N2c3ccc(-c4ccc(-c5ccccc5)cc4)cc3C(C)(C)c3c2c1cc1c3sc2ccccc21 ; c1ccc(-c2ccc3c(c2)C(c2ccccc2)(c2ccccc2)c2cc4c(sc5ccccc54)c4c2N3c2ccc(-c3ccccc3)cc2C4(c2ccccc2)c2ccccc2)cc1]

18. The organic EL device of claim 11, wherein the material represented by Formula 1 is represented by one selected from Compounds 21 to 27:

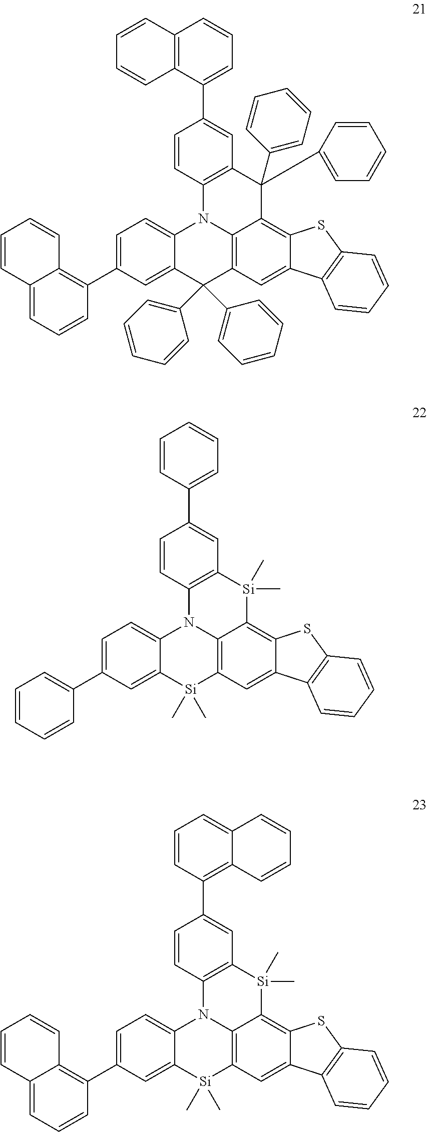 [SMILES: c1ccc(C2(c3ccccc3)c3cc(-c4cccc5ccccc45)ccc3N3c4ccc(-c5cccc6ccccc56)cc4C(c4ccccc4)(c4ccccc4)c4c3c2cc2c4sc3ccccc32)cc1 ; C[Si]1(C)c2cc(-c3ccccc3)ccc2N2c3ccc(-c4ccccc4)cc3[Si](C)(C)c3c2c1cc1c3sc2ccccc21 ; C[Si]1(C)c2cc(-c3cccc4ccccc34)ccc2N2c3ccc(-c4cccc5ccccc45)cc3[Si](C)(C)c3c2c1cc1c3sc2ccccc21]

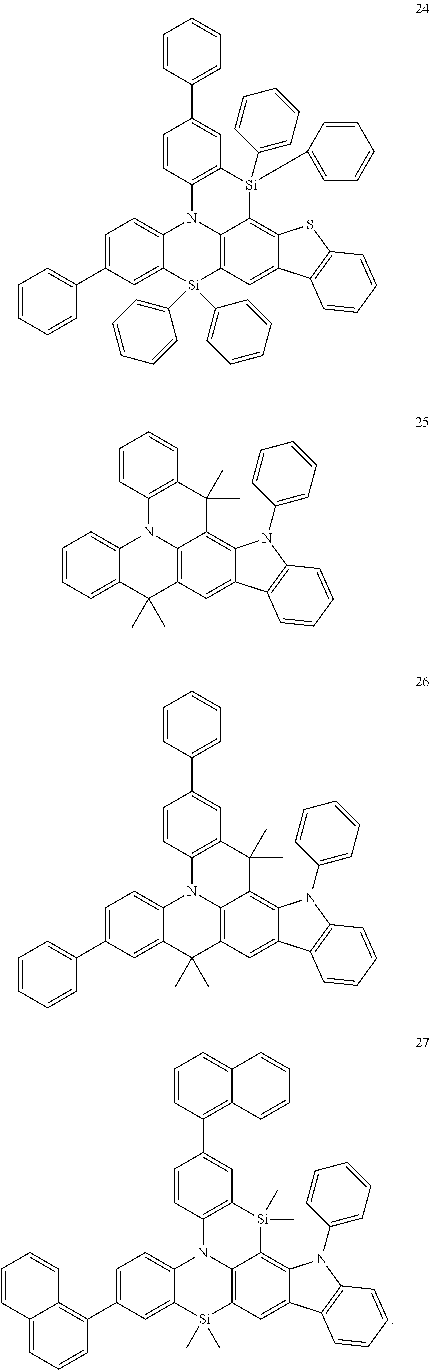 [SMILES: c1ccc(-c2ccc3c(c2)[Si](c2ccccc2)(c2ccccc2)c2cc4c(sc5ccccc54)c4c2N3c2ccc(-c3ccccc3)cc2[Si]4(c2ccccc2)c2ccccc2)cc1 ; CC1(C)c2ccccc2N2c3ccccc3C(C)(C)c3c2c1cc1c2ccccc2n(-c2ccccc2)c31 ; CC1(C)c2cc(-c3ccccc3)ccc2N2c3ccc(-c4ccccc4)cc3C(C)(C)c3c2c1cc1c2ccccc2n(-c2ccccc2)c31 ; C[Si]1(C)c2cc(-c3cccc4ccccc34)ccc2N2c3ccc(-c4cccc5ccccc45)cc3[Si](C)(C)c3c2c1cc1c2ccccc2n(-c2ccccc2)c31]

19. The organic EL device of claim 11, wherein the material represented by Formula 1 is represented by selected from Compounds 28 to 33:

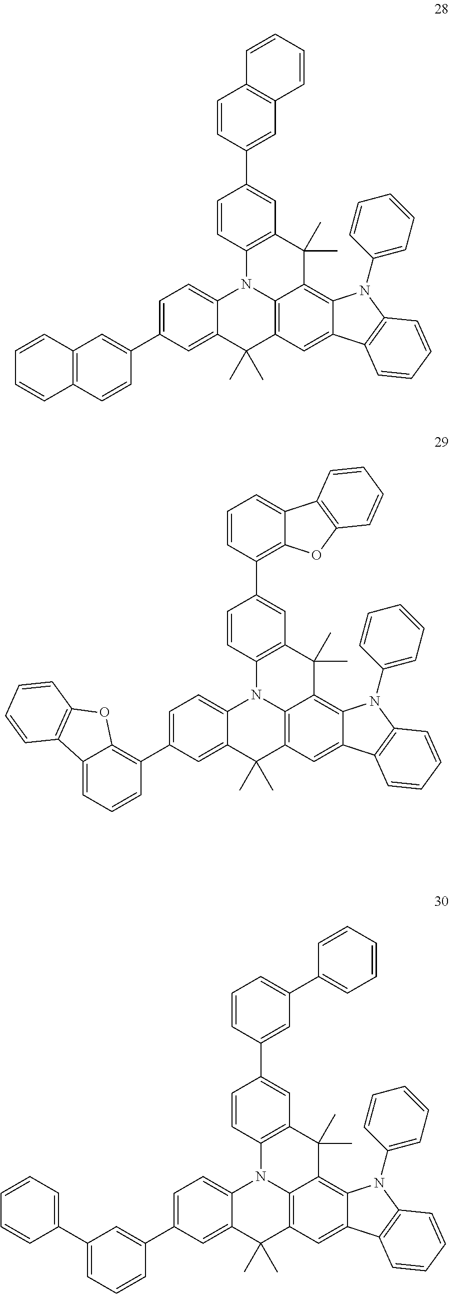 [SMILES: CC1(C)c2cc(-c3ccc4ccccc4c3)ccc2N2c3ccc(-c4ccc5ccccc5c4)cc3C(C)(C)c3c2c1cc1c2ccccc2n(-c2ccccc2)c31 ; CC1(C)c2cc(-c3cccc4c3oc3ccccc34)ccc2N2c3ccc(-c4cccc5c4oc4ccccc45)cc3C(C)(C)c3c2c1cc1c2ccccc2n(-c2ccccc2)c31 ; CC1(C)c2cc(-c3cccc(-c4ccccc4)c3)ccc2N2c3ccc(-c4cccc(-c5ccccc5)c4)cc3C(C)(C)c3c2c1cc1c2ccccc2n(-c2ccccc2)c31]

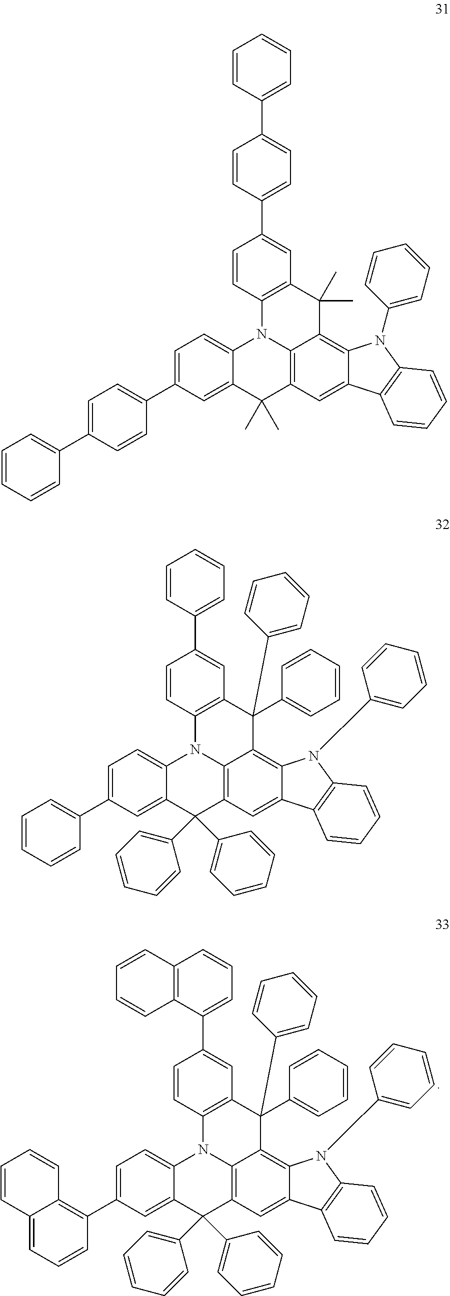 [SMILES: CC1(C)c2cc(-c3ccc(-c4ccccc4)cc3)ccc2N2c3ccc(-c4ccc(-c5ccccc5)cc4)cc3C(C)(C)c3c2c1cc1c2ccccc2n(-c2ccccc2)c31 ; c1ccc(-c2ccc3c(c2)C(c2ccccc2)(c2ccccc2)c2cc4c5ccccc5n(-c5ccccc5)c4c4c2N3c2ccc(-c3ccccc3)cc2C4(c2ccccc2)c2ccccc2)cc1 ; c1ccc(-n2c3ccccc3c3cc4c5c(c32)C(c2ccccc2)(c2ccccc2)c2cc(-c3cccc6ccccc36)ccc2N5c2ccc(-c3cccc5ccccc35)cc2C4(c2ccccc2)c2ccccc2)cc1]

20. The organic EL device of claim 11, wherein the material represented by Formula 1 is represented by one selected from Compounds 34 to 39: 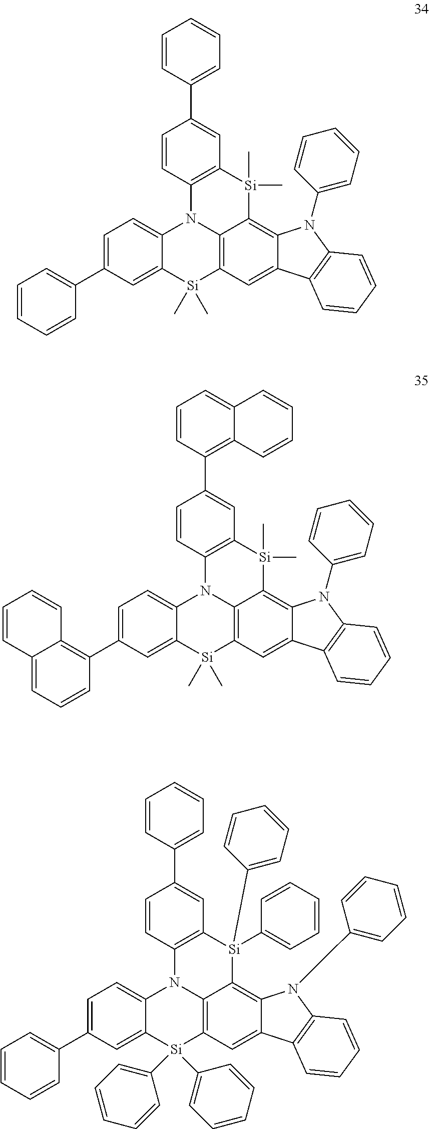 [SMILES: C[Si]1(C)c2cc(-c3ccccc3)ccc2N2c3ccc(-c4ccccc4)cc3[Si](C)(C)c3c2c1cc1c2ccccc2n(-c2ccccc2)c31 ; C[Si]1(C)c2cc(-c3cccc4ccccc34)ccc2N2c3ccc(-c4cccc5ccccc45)cc3[Si](C)(C)c3c2c1cc1c2ccccc2n(-c2ccccc2)c31 ; c1ccc(-c2ccc3c(c2)[Si](c2ccccc2)(c2ccccc2)c2cc4c5ccccc5n(-c5ccccc5)c4c4c2N3c2ccc(-c3ccccc3)cc2[Si]4(c2ccccc2)c2ccccc2)cc1] [chemical structure]
